(19) United States
(12) Patent Application Publication
(10) Pub. No.: US 2026/0114999 A1
(43) Pub. Date: Apr. 30, 2026

(54) HYBRID ENERGY STORAGE IN AN IMPLANTABLE MEDICAL DEVICE

(71) Applicant: Boston Scientific Scimed, Inc., Maple Grove, MN (US)

(72) Inventors: Kevin James Ivanca, Minneapolis, MN (US); Keith R. Maile, New Brighton, MN (US)

(21) Appl. No.: 19/364,318

(22) Filed: Oct. 21, 2025

Related U.S. Application Data
(60) Provisional application No. 63/712,638, filed on Oct. 28, 2024.

(51) Int. Cl. A61F 2/26; A61F 2/48

(52) U.S. Cl. CPC A61F 2/26; A61F 2/482

(57) ABSTRACT

An implantable fluid-operated device includes a rechargeable battery, a non-rechargeable battery, at least one capacitor, and a first piezoelectric pump for transferring fluid from the fluid reservoir to the inflatable member. A waveform of electrical energy is provided from the rechargeable battery to a piezoelectric element of the first piezoelectric pump to drive the first piezoelectric pump to pump fluid from the fluid reservoir to the inflatable member. The at least one capacitor is charged in response to a voltage generated by the piezoelectric element when the piezoelectric element returns to its neutral shape from a deformed shape. The provision of electrical energy to different groups of electrical circuits in the devices is controlled based on a state of charge of, for example, the at least one capacitor and the rechargeable battery.

### CROSS-REFERENCE TO RELATED APPLICATION

This application claims priority to U.S. Provisional Patent Application No. 63/715,638, filed on Oct. 28, 2024, entitled “HYBRID ENERGY STORAGE IN AN IMPLANTABLE MEDICAL DEVICE”, the disclosure of which is incorporated by reference herein in its entirety.

### TECHNICAL FIELD

This disclosure relates generally to bodily implants, and more specifically to hybrid energy storage techniques for use in an implantable medical device.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically-controlled manifold providing for the transfer of fluid within the implantable fluid-operated device.

The use of the electronic control system may provide for more accurate actuation and control of the flow of fluid between components of the inflatable device, thus improving performance and efficacy of the device, as well as patient comfort and safety. The electronic control system may include one or more electronically-operated pumps and one or more valves to control the flow of fluid in the system, and the pumps and valves may be operated by way of piezoelectric elements associated with the pumps and valves. Electronically-operated pumps and valves can be powered by an energy storage device (e.g., a battery) that is included in the implantable device. It is desirable for the implantable device to be small to avoid discomfort for the patient, and therefore the energy storage device should be small, but just large enough to power the components, including the pumps and valves, of the implantable device. However, when the energy storage device is small, a risk exists that it could be operated in a manner that places it at risk of failure.

### SUMMARY

In a general aspect, techniques described herein relate to an implantable fluid-operated device configured to control fluid flow between a fluid reservoir and an inflatable member. The device includes: a rechargeable battery configured for storing energy; energy transmission circuitry configured for receiving energy from an external power transmission device and for providing energy to charge the rechargeable battery; a non-rechargeable battery configured for storing energy; at least one capacitor configured for storing electrical charge; a first piezoelectric pump configured to transfer fluid from the fluid reservoir to the inflatable member; a first driver including first circuitry configured for providing a waveform of electrical energy from the rechargeable battery to a piezoelectric element of the first piezoelectric pump to drive the first piezoelectric to pump fluid from the fluid reservoir to the inflatable member, and second circuitry configured for charging the at least one capacitor in response to a voltage generated by the piezoelectric element when the piezoelectric element returns to its neutral shape from a deformed shape; a first group of at least one first electrical circuits; a second group of at least one second electrical circuits; a third group of at least one third electrical circuits; and a processor that is configured to execute instructions that cause the implantable fluid-operated device to: switch a provision of electrical energy to the first group between the at least one capacitor and the rechargeable battery based on a state of charge of the at least one capacitor; control whether electrical energy is provided to the second group from the rechargeable battery based on a state of charge of the rechargeable battery; and switch the provision of electrical energy to the third group between the rechargeable battery and the non-rechargeable battery based on a state of charge of the rechargeable battery.

Implementations can include one or more of the following features, alone or in any combination with each other.

For example, the processor can be configured to execute instructions that cause the implantable fluid-operated device to provide electrical energy to the first group from the at least one capacitor when a state of charge on the at least one capacitor exceeds a first threshold and to provide electrical energy to the first group from the rechargeable battery when the state of charge is less than the first threshold.

In another example, the processor can be configured to execute instructions that cause the implantable fluid-operated device to provide electrical energy to the second group from the rechargeable battery when a state of charge of the rechargeable battery exceeds a second threshold and to not provide electrical energy to the second group from the rechargeable battery when the state of charge of the rechargeable battery is less than the second threshold.

In another example, the processor can be configured to execute instructions that cause the implantable fluid-operated device to provide electrical energy to the second group and to the third group from the rechargeable battery when a state of charge of the rechargeable battery exceeds the second threshold and to not provide electrical energy to the second group from the rechargeable battery when the state of charge of the rechargeable battery is less than the second threshold and to provide electrical energy to the third group from the non-rechargeable battery when the state of charge of the rechargeable battery is less than a third threshold.

In another example, the third threshold can be equal to the second threshold.

In another example, the first driver and the second circuitry can belong to the second group.

In another example, the second group can include the first group.

In another example, the implantable fluid-operated device can include a penile implant, and the implantable fluid-operated device can further include: a second piezoelectric pump configured to transfer fluid from the inflatable member to the fluid reservoir; a second driver including third circuitry configured for providing a waveform of electrical energy from the rechargeable battery to a piezoelectric element of the second piezoelectric pump to drive the second piezoelectric to pump fluid from the inflatable member to the fluid reservoir, and fourth circuitry configured for charging the at least one capacitor in response to a voltage generated by the piezoelectric element when the piezoelectric element returns to its neutral shape from a deformed shape; and a protection circuit configured to prevent the fourth circuitry from charging the rechargeable battery, where the second driver and the third and fourth circuitry belong to the second group.

In another example, the implantable fluid-operated device can further include one or more valve circuits configured for opening and closing one or more fluidic valves that control the flow of fluid between the fluid reservoir and the inflatable member, where the one or more valve circuits belong to the first group.

In another example, the inflatable member can include an inflatable cuff configured for implantation about a urethra of a patient, and the implantable fluid-operated device can further include: a second piezoelectric pump configured to transfer fluid from the inflatable member to the fluid reservoir; a second driver including third circuitry configured for providing a waveform of electrical energy from the rechargeable battery to a piezoelectric element of the second piezoelectric pump to drive the second piezoelectric to pump fluid from the inflatable member to the fluid reservoir, and fourth circuitry configured for charging the at least one capacitor in response to a voltage generated by the piezoelectric element when the piezoelectric element returns to its neutral shape from a deformed shape; and a protection circuit configured to prevent the fourth circuitry from charging the rechargeable battery, where the second driver and the third and fourth circuitry belong to the second group; one or more valve circuits configured for opening and closing one or more fluidic valves that control the flow of fluid between the fluid reservoir and the inflatable member, where the one or more valve circuits belong to the first group and to the third group.

In another example, the implantable fluid-operated device can further include: a Bluetooth® communication circuit that belongs to the second group; and a low power communication circuit configured for sending information to, and for receiving information from, an external controller, where the low power communication circuit belongs to the third group.

In some aspects, the techniques described herein relate to a method of operating an implantable fluid-operated device that controls fluid flow between a fluid reservoir and an inflatable member, where the device includes a rechargeable battery, a non-rechargeable battery, at least one capacitor, a first piezoelectric pump configured to transfer fluid from the fluid reservoir to the inflatable member. The method includes: providing, with a first driver that includes first circuitry, a waveform of electrical energy from the rechargeable battery to a piezoelectric element of the first piezoelectric pump to drive the first piezoelectric to pump fluid from the fluid reservoir to the inflatable member; charging, with second circuitry, the at least one capacitor in response to a voltage generated by the piezoelectric element when the piezoelectric element returns to its neutral shape from a deformed shape; switching a provision of electrical energy to a first group of at least one first electrical circuits from the at least one capacitor to the rechargeable battery based on a state of charge of the at least one capacitor; controlling whether electrical energy is provided to a second group of at least one second electrical circuits from the rechargeable battery based on a state of charge of the rechargeable battery; and switching the provision of electrical energy to a third group of at least one third electrical circuits from the rechargeable battery to the non-rechargeable battery based on a state of charge of the rechargeable battery.

Implementations can include one or more of the following features, alone or in any combination with each other.

For example, the method can further include: providing electrical energy to the first group from the at least one capacitor when a state of charge on the at least one capacitor exceeds a first threshold; and providing electrical energy to the first group from the rechargeable battery when the state of charge is less than the first threshold.

In another example, the method can further include: providing electrical energy to the second group from the rechargeable battery when a state of charge of the rechargeable battery exceeds a second threshold; and blocking the provision of electrical energy to the second group from the rechargeable battery when the state of charge of the rechargeable battery is less than the second threshold.

In another example, the method can further include providing electrical energy to the second group and to the third group from the rechargeable battery when a state of charge of the rechargeable battery exceeds the second threshold; blocking the provision of electrical energy to the second group from the rechargeable battery when the state of charge of the rechargeable battery is less than the second threshold; providing electrical energy to the third group from the non-rechargeable battery when the state of charge of the rechargeable battery is less than a third threshold.

In another example, the third threshold can be equal to the second threshold.

In another example, the first driver and the second circuitry can belong to the second group, and the second group can include the first group.

In another example, the implantable fluid-operated device can include a penile implant and a second piezoelectric pump configured to transfer fluid from the inflatable member to the fluid reservoir, and the method can further include: providing, with a second driver that includes third circuitry, a waveform of electrical energy from the rechargeable battery to a piezoelectric element of the second piezoelectric pump to drive the second piezoelectric to pump fluid from the inflatable member to the fluid reservoir, where the second driver and the third and fourth circuitry belong to the second group; charging, with fourth circuitry, the at least one capacitor in response to a voltage generated by the piezoelectric element when the piezoelectric element returns to its neutral shape from a deformed shape; and preventing the fourth circuitry from charging the rechargeable battery.

In another example, the implantable fluid-operated device can include one or more valve circuits configured for opening and closing one or more fluidic valves that control the flow of fluid between the fluid reservoir and the inflatable member, where the one or more valve circuits belong to the first group.

In another example, the inflatable member can include an inflatable cuff configured for implantation about a urethra of a patient, and the implantable fluid-operated device can further include a second piezoelectric pump configured to transfer fluid from the inflatable member to the fluid reservoir, and the method can further include: providing, by a second driver including third circuitry, a waveform of electrical energy from the rechargeable battery to a piezoelectric element of the second piezoelectric pump to drive the second piezoelectric to pump fluid from the inflatable member to the fluid reservoir, where the second driver belongs to the second group; charging, with fourth circuitry, the at least one capacitor in response to a voltage generated by the piezoelectric element when the piezoelectric element returns to its neutral shape from a deformed shape, where the third and fourth circuitry belong to the second group; controlling, with one or more valve circuits configured for opening and closing one or more fluidic valves, the flow of fluid between the fluid reservoir and the inflatable member, where the one or more valve circuits belong to the first group and to the third group; and preventing the fourth circuitry from charging the rechargeable battery.

### BRIEF DESCRIPTION OF THE DRAWINGS

FIG. 1 is a block diagram of an implantable fluid-operated inflatable device.

FIG. 2A illustrates a system including an example implantable fluid-operated inflatable device.

FIG. 2B illustrates a system including another example implantable fluid-operated inflatable device.

FIG. 3 is a schematic diagram of a fluidic architecture of an implantable fluid-operated inflatable device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 5A is an exploded view of an example pump device of a fluid control system of a fluid-operated inflatable device.

FIG. 5B is a cross-sectional view of the example pump device shown in FIG. 5A, in an open position.

FIGS. 6A, 6B, and 6C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 7 is a schematic block diagram of a system for driving a piezoelectric element of a piezoelectric-operated pump or valve and for monitoring and controlling the performance of the piezoelectric element.

FIG. 8A is a graph of the voltage amplitude of an example waveform that can be a provided by the driver to the piezoelectric element to drive the piezoelectric element to cause a pump associated with the piezoelectric element to pump fluid.

FIG. 8B is a graph of the voltage amplitude of another example waveform that can be a provided by the driver to the piezoelectric element to drive the piezoelectric element to cause a pump associated with the piezoelectric element to pump fluid.

FIG. 9 is an example schematic diagram of a system for driving electrically-operated pumps and valves of an implantable device including an inflatable member and for recovering energy from the pumps and valves to use for driving one or more of the pumps and valves.

FIG. 10A is an example graph of a current as a function of time, which is drawn from the rechargeable battery by a driver that drives a piezoelectric pump when there is no protection circuit between the battery and the driver.

FIG. 10B is an example graph of a current as a function of time, which is drawn from the rechargeable battery by a driver that drives a piezoelectric pump when a protection circuit 904 is connected between the battery and the driver.

FIG. 11 is an example schematic diagram of a system for driving electrically-operated pumps and valves of an implantable device including an inflatable member and for recovering energy from the pumps and valves to use for driving one or more of the pumps and valves.

FIG. 12 is an example schematic diagram of a system that includes processor-controlled switches that couple piezoelectric drivers to a battery or to the one or more capacitors.

FIG. 13 is an example schematic diagram of an implantable fluid-operated medical device system that includes a plurality of different energy storage devices that are configured for storing energy and providing electrical energy to one or more electrical circuits of the system.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically-operated.

For example, the pumps and/or valves of the fluid control system can be electronically-operated by the fluid control system to control the pressure of, and the flow of fluid in, parts of the fluid-operated inflatable device. An electronically-operated fluid control system, in accordance with implementations described herein, can include a plurality of electromechanical devices, such as piezoelectric devices that operate as pumps or as valves in the system. One or more controllers can drive the electromechanical devices to perform their functions. The electromechanical devices and the one or more controllers can be powered by an energy storage device, such as a rechargeable battery. The rechargeable battery can be charged by an external charging system that transmits energy wirelessly to the rechargeable battery from outside the body in which the implantable device is implanted.

In some implementations, when the electromechanical devices are driven and powered with energy from the rechargeable battery, some amount of the energy is not consumed by the electromechanical devices and can be recovered for use at a later time. In some cases, the recovered energy is used to recharge the rechargeable battery. In some cases, the recovered energy is used to charge a capacitor, which can supply the energy stored on the capacitor for use in driving one or more of the electromechanical devices. Storing recovered energy on the capacitor, rather than in the rechargeable battery can help mitigate deleterious effects of micro charging of the battery on the long-term storage capacity of the battery.

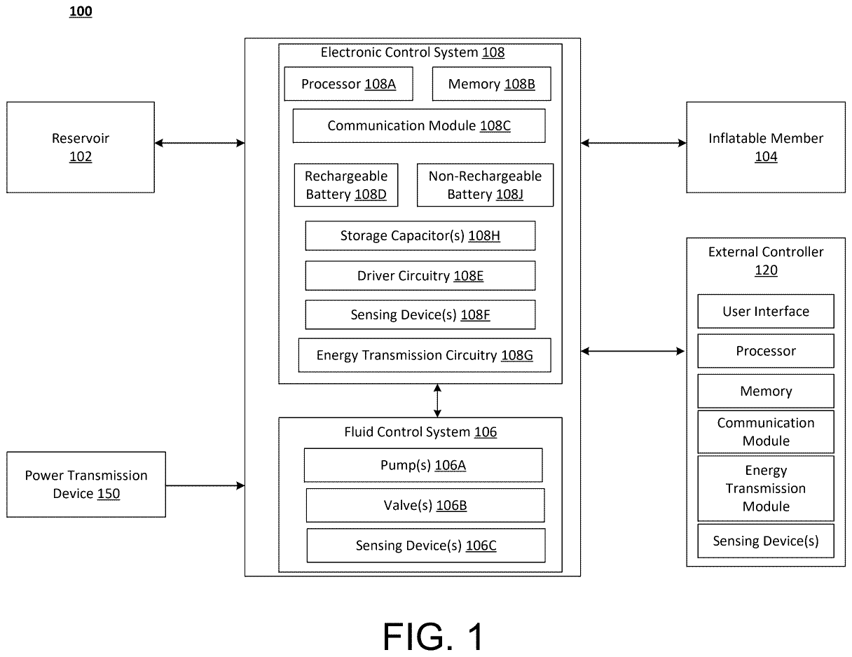

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidic components, such as one or more pumps 106A, one or more valves 106B, and the like configured to transfer fluid between the fluid reservoir 102 and the inflatable member 104. The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, a rechargeable battery 108D, a non-rechargeable battery 108J, one or more storage capacitors 108H, electronic driver circuitry 108E, sensing devices 108F, such as, for example, voltage measurement circuitry, current measurement circuitry, and energy transmission circuitry 108G. In some examples, the communication module 108C of the electronic control system 108 may provide for communication with one or more external devices such as, for example, an external controller 120. The rechargeable battery 108D can be recharged through the application of a current through the battery, and can be, for example, a lithium-ion battery, a NiCd battery, a NiMH battery, etc. The non-rechargeable battery 108J generally cannot be recharged through the application of a current through the battery without causing the release of significant amounts of gas that can damage the battery, and can include, for example, an alkaline battery, a lithium battery (e.g., an LiMnO₂ battery), etc.

In some examples, the external controller 120 includes components such as, for example, a user interface, a processor, a memory, a communication module, an energy transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like.

In some examples, the energy transmission module of the external controller 120 provides for charging of the components of the internal electronic control system 108. In some examples, transmission of energy for the charging of the internal electronic control system 108 can be, alternatively or additionally, provided by an external energy transmission device 150 that is separate from the external controller 120. In some implementations the external controller 120 can include sensing devices such as one or more pressure sensors, one or more accelerometers, and other such sensing devices.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

In some examples, electronic monitoring and control of the implantable fluid-operated inflatable device 100 may provide for improved patient control of the device, improved patient comfort, improved patient safety, and the like. In some examples, electronic monitoring and control of the implantable fluid-operated inflatable device 100 may afford the opportunity for tailoring of the operation of the inflatable device 100 by a physician without further surgical intervention. The fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

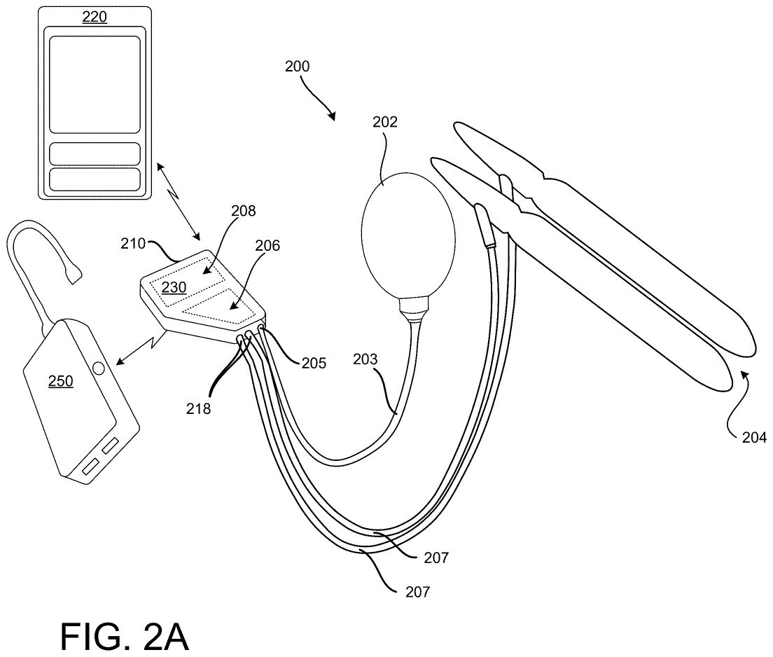

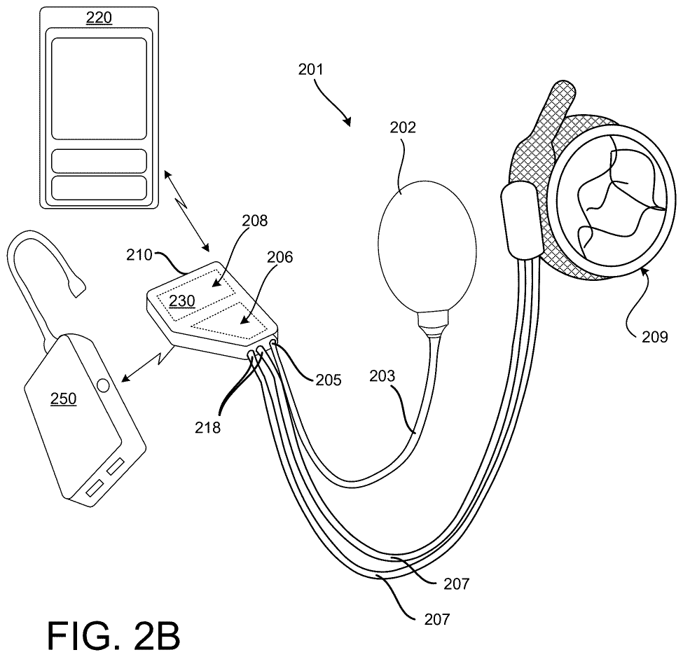

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2A or an inflatable artificial urinary tract sphincter as shown in FIG. 2B. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid-operated inflatable device 200 in the form of an example inflatable penile prosthesis is shown in FIG. 2A. Another example system including an example implantable fluid-operated inflatable device 201 in the form of an example artificial urinary tract sphincter is shown in FIG. 2B. The example implantable fluid-operated inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example implantable fluid-operated inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2A, the inflatable member 204 is in the form of a pair of inflatable cylinders, which are configured for implantation within the penis of a patient. In the example shown in FIG. 2B, the inflatable member 209 is in the form of an inflatable cuff that is configured for implantation around the urethra of a patient. In the examples shown in FIGS. 2A and 2B, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204 or the inflatable member 209.

In the example shown in FIG. 2A, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 218 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the implantable fluid-operated inflatable device 200. In some examples, components of the electronic control system 208 and/or the fluid control system 206 can be charged and/or recharged by an energy transmission module of the external controller 220, and/or by an energy transmission device 250, that is separate from the external controller 220. The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204.

The principles to be described herein are applicable to the example implantable fluid-operated inflatable device, in the form of the example inflatable penile prostheses shown in FIG. 2A, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. For example, as shown in FIG. 2B, the inflatable member 209 can include an inflatable cuff, which may be implemented as an artificial urinary sphincter. The inflatable cuff 209 is or may be disposed about a urethra proximate the bladder. The implantable fluid-operated inflatable device 201 can be activated to pump fluid from a reservoir to expand the cuff 209 and to close the urethra. The cuff 209 is deflated to allow a patient to void the bladder.

As noted above, the electronic control system 208 controlling the flow of fluid between the reservoir 202 and the inflatable member 204 for inflation, pressurization, deflation, depressurization and the like of the inflatable member 204 may provide for improved patient control of the implantable fluid-operated inflatable device 200, improved accuracy in operation of the implantable fluid-operated inflatable device 200, improved patient comfort, improved patient safety, and the like. In some situations, this improved control and improved accuracy in the operation of the implantable fluid-operated inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or one or more valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

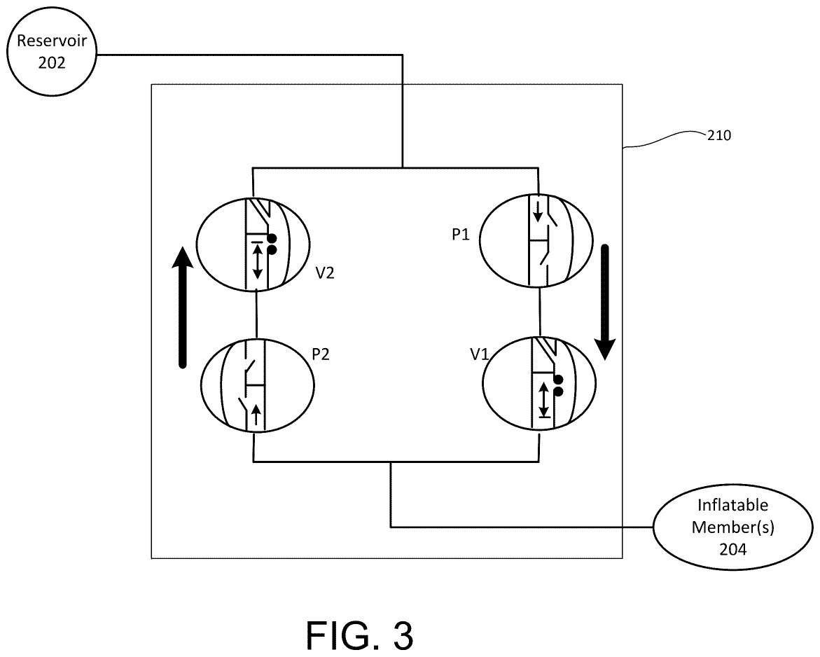

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

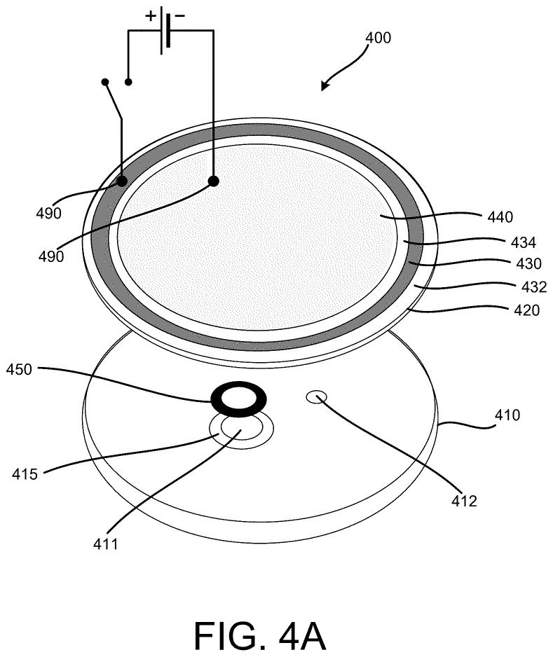

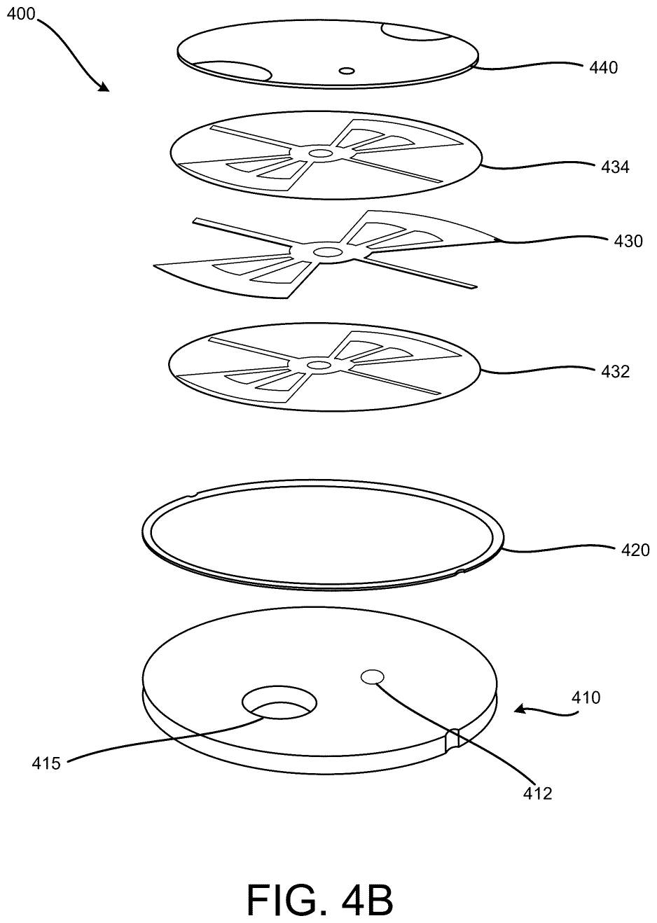

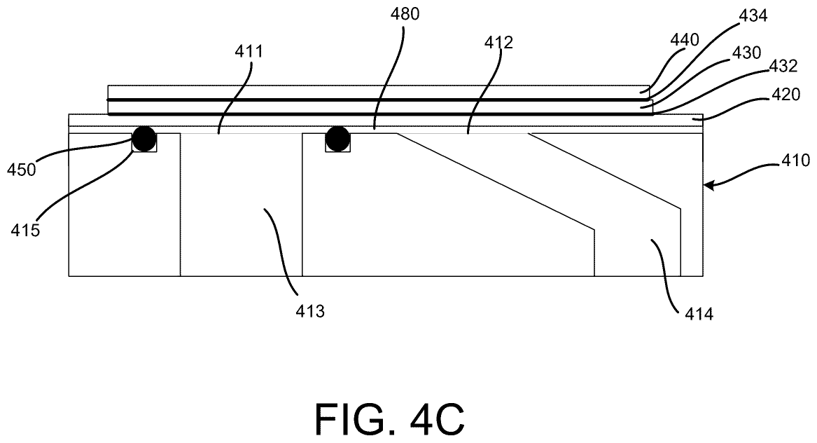

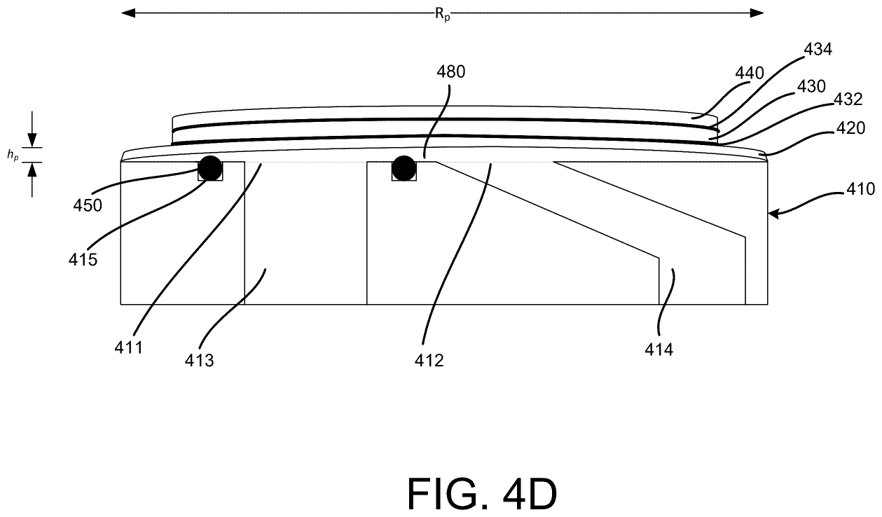

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. The example valve device 400 shown in FIGS. 4A-4D is an example of a fluid control device, or a fluidic component, included in the fluid control system 206 of the example electronically controlled fluid manifold 230 described above.

In the example arrangement shown in FIGS. 4A-4D, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery of in the implantable fluid-operated inflatable device 100) to drive the diaphragm 420 to open and close the valve device 400. The diaphragm 420 can include a thin metal foil, whose shape can be repeatably deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 10¹³ Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. For example, in some implementations, the diaphragm 420 can be bonded to the base plate 410 at the periphery of the diaphragm to form a fluid-tight connection between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480. In the case of a circular diaphragm 420, the fluid chamber 480 can have a radius, Rp, and a height, hp, that depends on the voltage of the piezoelectric element 440 that is actuated to change the shape of the diaphragm.

The general architecture and principles of operation of the valve device described above also can be used to implement one or more pumps (such as pumps P1, P2 of FIG. 3) to pump fluid from one location to another. For example, repeated movement of a diaphragm between an open position and a closed position, relative to a base plate, can cause fluid to be drawn into a chamber formed between the diaphragm and the base plate through a first fluid passageway and expelled out of the chamber into a second fluid passageway. In this manner, fluid can be pumped from a first location that is fluidically connected to the first passageway to a second location that is fluidically connected to the second passageway. In some implementations, one or more one-way valves can be configured to prevent, or limit, the flow of fluid in the direction from the second location to the first location.

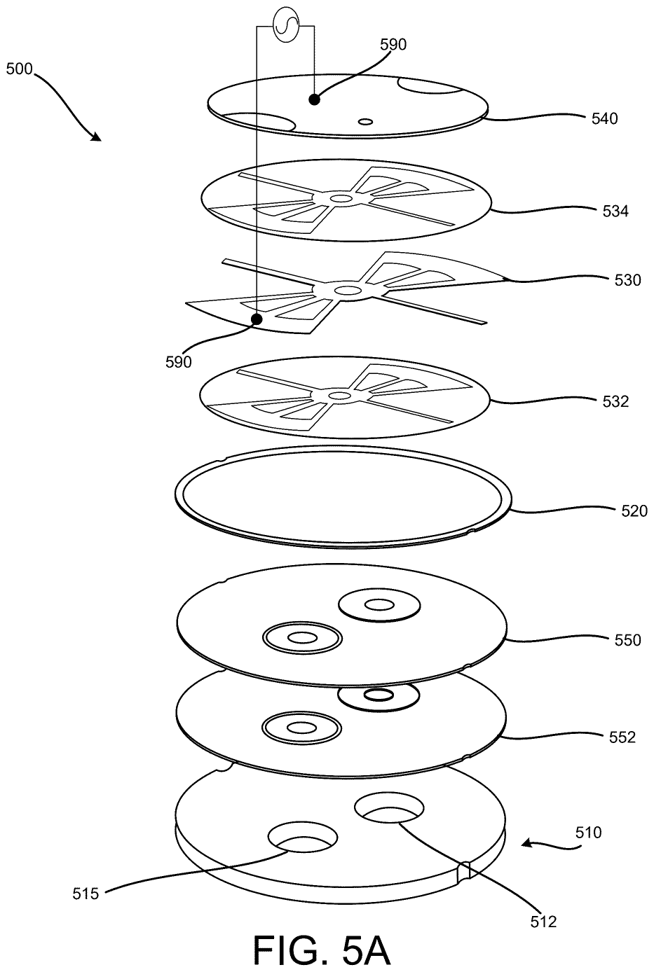

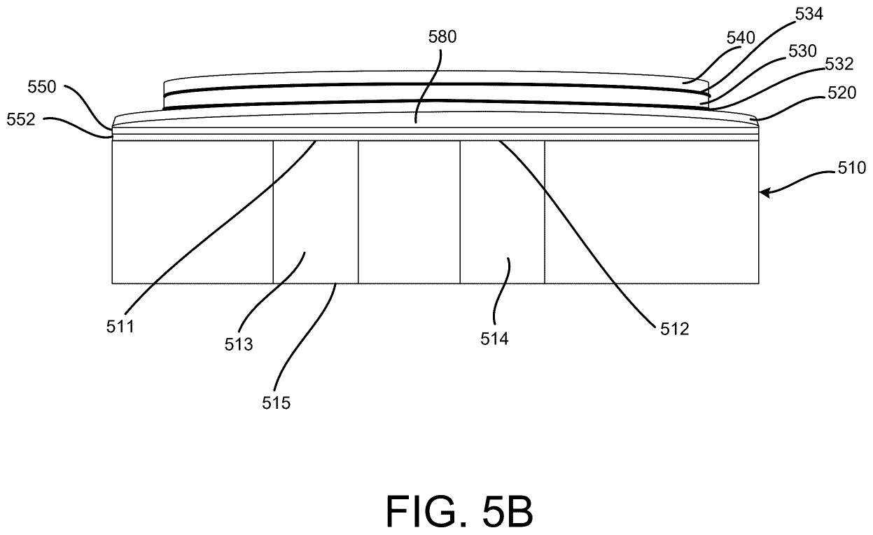

FIG. 5A is a partially exploded perspective view of an example pump device 500, and FIG. 5B is a cross-sectional view of the example pump device 500. The example pump device 500 shown in FIGS. 5A-5B is an example of a fluid control device, or a fluidic component, included in the fluid control system 206 of the example electronically controlled fluid manifold 230 described above.

In the example arrangement shown in FIGS. 5A-5B, the example pump device 500 includes a base plate 510 defining a base portion of the pump device 500. A diaphragm 520 is positioned on the base plate 510. A piezoelectric element 540 is positioned on the diaphragm 520, with an isolation layer 530 positioned between the diaphragm 520 and the piezoelectric element 540. The piezoelectric element can be electrically powered (e.g., by a battery of the implantable fluid-operated inflatable device 100) to drive the diaphragm 520 to pump fluid through the pump device 500. The diaphragm 520 can include a thin metal foil, whose shape can be repeatably deformed in response to movement by the piezoelectric element 540. In some implementations, the diaphragm 520 can include titanium material. In some implementations, the diaphragm 520 can include gold material. In some implementations, the diaphragm 520 can include stainless steel material or other alloys. In some implementations, the isolation layer 530 can include a polyamide material that has a high resistivity, for example, a resistivity greater than 10¹³ Ohm-cm to provide electrical isolation between the piezoelectric element 540 and the diaphragm 520.

In some examples, an epoxy layer 532 provides for the coupling of the isolation layer 530 and the diaphragm 520. In some examples, an epoxy layer 534 provides for the coupling of the piezoelectric element 540 and the isolation layer 530, and the epoxy layers 532, 534 together provide for the coupling of the piezoelectric element 540 to the diaphragm 520. In some implementations, the epoxy layers 532, 534 are not distinct but are part of one epoxy layer.

In some examples, one or more electrodes 590 are arranged on the example pump device 500. In the example shown in FIG. 5A, the example pump device 500 includes a pair of electrodes 590 coupled between the isolation layer 530 and the piezoelectric element 540. Application of a voltage to the piezoelectric element 540 causes a deflection or deformation of the piezoelectric element 540 and a corresponding deflection or deformation of the diaphragm 520 coupled thereto.

When the pump device 500 is used in the fluid control system 206 of the example electronically controlled fluid manifold 230 described above, the piezoelectric element 540 can be controlled to cause fluid to be pumped by device 500, for example, by repeatedly changing a volume of the fluid chamber 580 by deforming the deformable diaphragm 520 to pump fluid from the fluid reservoir to the inflatable member.

In the example arrangement shown in FIGS. 5A-5B, a fluid chamber 580 is defined between the base plate 510 and the diaphragm 520. The base plate 510 includes a first opening 515 that provides for communication between a first fluid passageway 513 and the fluid chamber 580. The base plate 510 includes a second opening 512 that provides for communication between a second fluid passageway 514 and the fluid chamber 580. In some examples, the diaphragm 520 can be actuated to move between a closed position in which the diaphragm 520 is proximate to the base plate 510 due to the deflection of the diaphragm 520, such that the volume of the chamber 580 is minimized, and an open position in which the base plate 510 is separated, or spaced apart from, the diaphragm 520 due to the deflection of the diaphragm 520, such that the volume of the chamber is maximized. When the diaphragm 520 is actuated to move from the closed position to the open position, fluid can be drawn into the chamber 580 through the first fluid passageway 513, and when the diaphragm 520 is actuated to move from the open position to the closed position, fluid can be expelled from the chamber 580 through the second fluid passageway 514. Repeatedly actuating the diaphragm between the closed and open position allows fluid to be pumped through the pump device 500, from the first fluid passageway 513 to the second fluid passageway 514 via the fluid chamber 580.

In some implementations, the pump device 500 can include one or more foil plates 550 and 552 to control the flow of fluid into and out of the pump device 500. The foil plates 550, 552 can include one-way check valves that operate to permit fluid to flow in one direction through the values but not in an opposite direction. The one-way check valves defined by the one or more foil plates can be positioned in, or in fluid connection with, a fluid passageway 513, 514 of the pump device 500. In some examples, a check valve is positioned in, or in fluid connection with, a portion of a fluid passageway 513, 514 so as to inhibit the unintended flow of fluid through the pump device in the event of a fluctuation, or spike in pressure. In some examples, a check valve is positioned in a fluid passageway 513, 514 so as to counteract a back pressure that would otherwise overcome the closing pressure and cause unintentional flow through the pump device 500. In some example implementations, a first check valve defined by one or more foil plates 550, 552 is positioned in, or in fluid connection with (e.g., at a first opening 511 of), a first fluid passageway 513 of the pump device and is configured to permit fluid to easily flow from the first fluid passageway 513 into the chamber 580 but to prevent or inhibit the flow of fluid from the chamber 580 into the passageway 513. In some example implementations, a second check valve defined by one or more foil plates 550, 552 is positioned in, or in fluid connection with (e.g., at a first opening 512 of), a second fluid passageway 514 of the pump device 500 and is configured to permit fluid to easily flow from the chamber 580 into the second fluid passageway 513 but to prevent or inhibit the flow of fluid from the passageway 513 into the chamber 580.

Application of an alternating current (AC) voltage to the piezoelectric element 540 can cause the diaphragm 520 of the pump device 500 to oscillate between a first position that defines the closed position of the chamber 580, in which the diaphragm 520 is proximate to the base plate 510 and the volume of the chamber 580 is minimized, and a second (e.g., domed) position that defines the open position of the chamber 580, in which the diaphragm 520 is separated from the base plate and the volume of the chamber 580 is maximized. As the diaphragm 520 of the pump device 500 oscillates between a first position and the second position, fluid is drawn into the chamber 580 from the first passageway 513 and is expelled from the chamber 580 into the second passageway 514. As the diaphragm 520 of the pump device 500 oscillates between a first position and the second position, the one-way check valves defined by the one or more foil plates 550, 552 prevent or inhibit fluid from flowing from the chamber 580 into the first passageway 513 and prevent or inhibit fluid from flowing into the chamber 580 from the second passageway 514. Thus, the application of the AC voltage to the piezoelectric element 540 causes the pump device 500 to pump fluid from the first passageway 513 to the second passageway 514.

The piezoelectric element 540 can be controlled to cause fluid to be pumped by device 500, for example, by repeatedly changing a volume of the fluid chamber 580 by deforming the deformable diaphragm 520 to pump fluid from the fluid reservoir to the inflatable member.

The volume of the chamber 580 can be determined, at least in part, by the shape, geometry, and material properties of the components used to form the chamber 580, including, for example, the base plate 510 and the deformable diaphragm 520. In some cases, a relatively larger volume of the chamber 580, for an approximately constant diameter of the chamber, can result in more fluid being pumped in each open/close cycle of the pump device 500. To achieve a relatively larger volume of chamber 580, the deformable diaphragm can be deformed or biased into a non-flat dome-shaped configuration before it is attached to the piezoelectric element 540.

In some implementations, before the diaphragm 520 is attached to the piezoelectric element 540, a voltage can be placed across the electrodes 590 attached to the piezoelectric element 540 to configure the piezoelectric element 540 in the domed configuration that it assumes when the fluid chamber is in the open position (See FIG. 4D). Then, the diaphragm can be placed in contact with the piezoelectric element while the piezoelectric element 440 is in its domed configuration, and the epoxy can be cured when the piezoelectric element and the diaphragm 420 are in the domed configuration, which can reduce stress on the adhesive bond between the diaphragm 420 and the piezoelectric element 440.

Referring again to FIGS. 2A and 2B, although considerable effort is expended to maintain the cleanliness of the components of the system and the purity of the fluid used within the system, it is still possible that some small amounts of foreign matter can contaminate the fluid within the system. For example, when the reservoir 202, the inflatable members 204, and the housing 210 are implanted and connected (e.g., by conduits 203, 207) within a patient, it is possible that some contamination enters the fluidic system. In addition, it is possible that, once implanted within a patient, that small amounts of material disintegrate from walls of the reservoir 202, inflatable member 204, housing 210 and conduits 203, 207 and become suspended within fluid that flows within the implantable fluid-operated inflatable device 200. Because of the small internal dimensions of the pumps and valves used within the fluidic system, the existence of particles of foreign matter suspended within the fluid flowing within the system poses a risk of clogging or damaging one or more of the pumps and valves, which may lead to malfunction of the implantable fluid-operated inflatable device 200. To mitigate the effect of any particulate matter suspended within the fluid that flows within the implantable fluid-operated inflatable device 200, the fluidic path can include one or more filters that block, or reduce the amount of, particulate matter that enters the pumps and valves of the system. In some implementations, the filters can be included in a fluid pathway of a pump or valve.

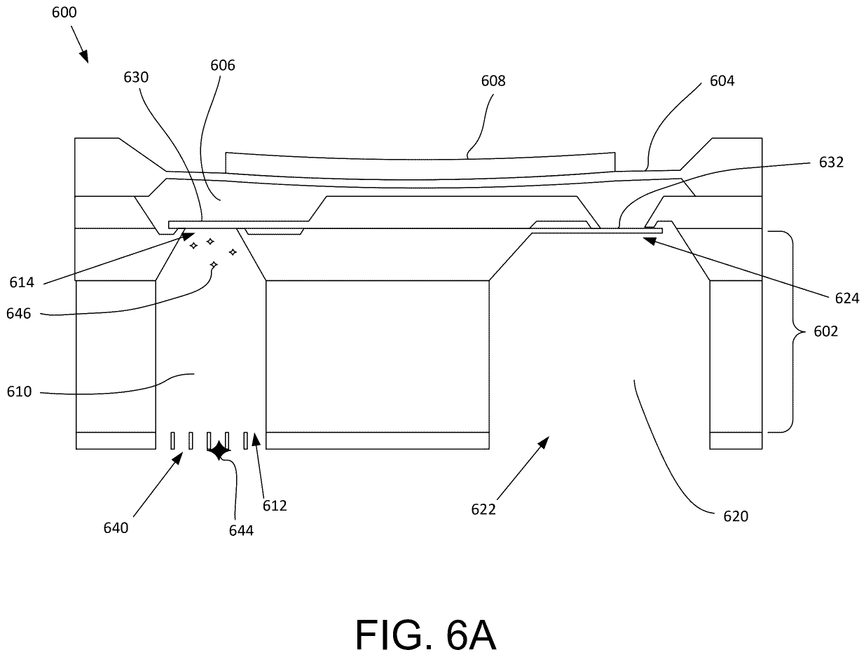

FIGS. 6A, 6B, 6C are cross-sectional views of example pump devices 600 that include a filter for capturing particulate matter in the fluid flow and/or for blocking the particulate matter from entering certain parts of the fluidic system (e.g., for blocking particulate matter from entering a pump chamber of the device). The example pump device 600 shown in FIGS. 6A, 6B, 6C are examples of a fluid control device, or a fluidic component, included in the fluid control system 206 of the example electronically controlled fluid manifold 230 described above.

In the example arrangements shown in FIGS. 6A, 6B, 6C the example pump device 600 includes a base plate 602 defining a base portion of the pump device 600. A diaphragm 604 is positioned above the base plate 602, and a fluid chamber 606 is defined between the base plate 602 and the diaphragm 604. A piezoelectric element 608 is positioned on the diaphragm 604. The piezoelectric element can be electrically powered (e.g., by a rechargeable battery of the implantable fluid-operated inflatable device) to drive the diaphragm 604 to pump fluid through the pump device 600. The diaphragm 604 can include a thin metal foil, whose shape can be repeatably deformed in response to movement by the piezoelectric element 608. In some implementations, the diaphragm 604 can include titanium material.

The base plate 602 can define a first fluid passageway 610 through which fluid can flow from a fluid reservoir into the fluid chamber 606. The first fluid passageway 610 can include an opening 612 at a first end of the passageway 610, which is distal to the fluid chamber 606, and can include an opening 614 and a second end of the passageway 610, which is proximate to the fluid chamber 606. The base plate 602 can define a second fluid passageway 620 through which fluid can flow from the fluid chamber 606 to an inflatable member. The second fluid passageway 620 can include an opening 622 at a first end of the passageway 620, which is distal to the fluid chamber 606, and can include an opening 624 and a second end of the passageway 620, which is proximate to the fluid chamber 606. In some implementations, the first fluid passageway 610 and the second fluid passageway 620 can be tapered, such the passageways 610, 620 have larger cross-sectional areas at the ends 612, 622 of the passageways that are distal to the fluid chamber 606 than at ends of the passageways that are proximate to the fluid chamber.

The pump device 600 can include a first flexible flap 630 that includes a portion that has an area that is greater than an area of the passageway opening 614 that is proximate to the fluid chamber 606 and that covers the opening, such that the first flexible flap 630 is configured to seal against portions of the base plate that defines the opening 614 of the first fluid passageway 610 to close the opening 614 when a fluid pressure in the fluid chamber 606 is greater than a fluid pressure of fluid in the first fluid passageway 610. The flexible flap 630 can be secured to the base plate over a portion of its extent but can have a portion that is unsecured, such that at least a portion of the flexible flap is configured to be pushed away from one or more walls of the fluid passageway 610 that defines the opening 614 when a fluid pressure of fluid in the first fluid passageway 610 is greater than a fluid pressure in the fluid chamber 606. In this manner, the flexible flap 630 operates to allow fluid to flow from the first fluid passageway 610 into the fluid chamber 606 but to block the flow of fluid from the fluid chamber 606 into the first fluid passageway 610. The flexible flap 630 can be made of a variety of materials including, for example, titanium, elastomeric material, plastic material, etc.

The pump device 600 can include a second flexible flap 632 that includes a portion that has an area that is greater than an area of the passageway opening 624 that is proximate to the fluid chamber 606 and that covers the opening, such that the second flexible flap 632 is configured to seal against portions of the base plate that defines the opening 624 of the second fluid passageway 620 to close the opening 624 when a fluid pressure in the fluid chamber 606 is greater than a fluid pressure of fluid in the second fluid passageway 620. The flexible flap 632 can be secured to the base plate over a portion of its extent but can have a portion that is unsecured, such that at least a portion of the flexible flap is configured to be pushed away from one or more walls of the second fluid passageway 620 that defines the opening 624 when a fluid pressure in the fluid chamber 606 is greater than a fluid pressure of fluid in the second fluid passageway 620. In this manner, the flexible flap 632 operates to allow fluid to flow from the fluid chamber 606 into the second fluid passageway 620 but to block the flow of fluid from the second fluid passageway 620 into the fluid chamber 606. The flexible flap 632 can be made of a variety of materials including, for example, titanium, elastomeric material, plastic material, etc.

With the flexible flaps 630, 632 configured in this way to allow fluid to flow in a first direction from the first fluid passageway 610 into the fluid chamber 606 and out of the fluid chamber into the second fluid passageway 620 but not in a direction opposite to the first direction, repeated expansion and contraction of the volume of the fluid chamber 606 in response to the piezoelectric element 608 operating on the deformable diaphragm 604 can cause fluid to be pumped from a reservoir fluidically connected to the first fluid passageway 610 to an inflatable member that is fluidically connected to the second fluid passageway 620.

The pump device 600 can include a fluid filter 640 that is located within, or at the end 612 of, the first fluid passageway 610 or that is located within, or at the end 622 of, the second fluid passageway 620. The fluid filter 640 can operate to block, for example, debris, foreign matter, particulates suspended in the fluid flowing through the device 600 from passing through the first fluid passageway 610 and into the fluid chamber 606 and/or from exiting the second fluid passageway 620. For example, as shown in FIG. 6A, a fluid filter 640 is located at the opening 612 into the first fluid passageway 610. As shown in FIG. 6B, a fluid filter 640 is located at the opening 622 into the second fluid passageway 620. As shown in FIG. 6C, a fluid filter 640A is located at the opening 612 into the first fluid passageway 610, and a fluid filter 640B is located at the opening 622 into the second fluid passageway 620.

In some implementations, the fluid filter 640, 640A, 640B can include a metal foil (e.g., a titanium foil, having a pattern of openings that permit fluid to flow through the openings but that block particulates having a characteristic size larger than a threshold size from flowing through the opening. For example, particulates 644 having a characteristic size (e.g., minimum transverse extent) that is greater than a threshold size defined by the size (e.g., diameter) of the openings can be blocked by the filter 640, while particulates 646 and a characteristic size smaller than the threshold size can pass through the filter 640.

The example pump devices 600 shown in FIGS. 6A, 6B, 6C include filters 640, 640A, 640B for blocking particulate matter in the fluid from entering a pump chamber of the device or for circulating in the fluidic system in which the pump devices operate. The filter 640 shown in FIG. 6A is disposed at the distal end 612 of the first fluid passageway 610, and the filter 640 shown in FIG. 6B is disposed at the distal end 622 of the second fluid passageway 620. These filters 640 can be similar to the filters 640, 640B, 640B shown in FIGS. 6A-10, in that the filters 640 can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 600 shown in FIGS. 6A, 6B, 6C can include filters disposed within the first fluid passageway 610 or within the second fluid passageway 620, for example, between the first end 612 of the first fluid passageway 610 and the opening 614 at the second end of the first fluid passageway 610 and/or between the first end 622 of the second fluid passageway 620 and the opening 624 at the second end of the second fluid passageway 620. For example, as shown in FIG. 6A, the example pump device 600 can include a filter 640 disposed within the first fluid passageway 610. In another example, as shown in FIG. 6B, the example pump device 600 can include a filter 640 disposed within the second fluid passageway 620. In another example, as shown in FIG. 6C, the example pump device 600 can include a filter 640A disposed within the first fluid passageway 610 and another filter 640B disposed within the second fluid passageway 620.

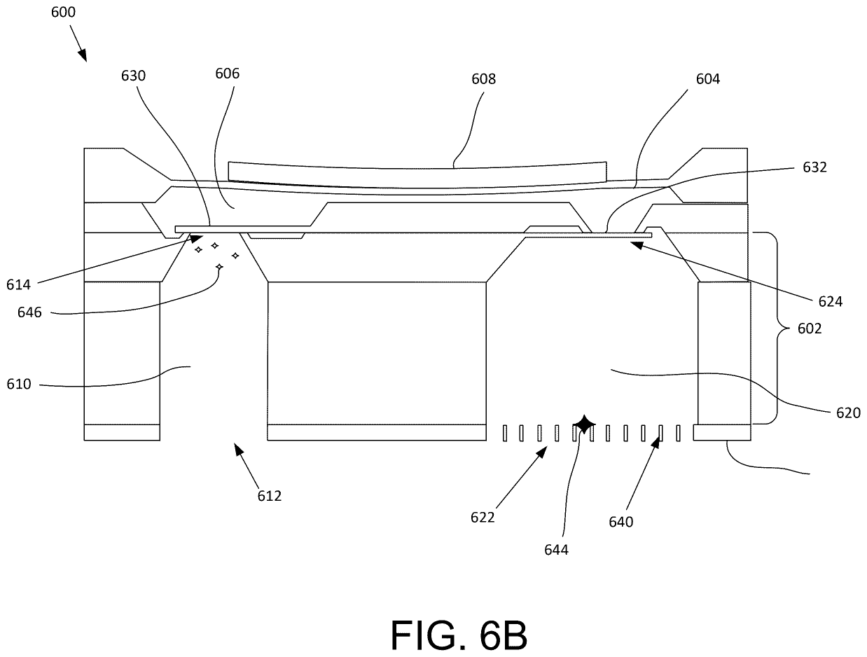

FIG. 7 is a schematic block diagram of an implantable fluid-operated system 700 for driving a piezoelectric element 714 of a piezoelectric-operated pump or valve and for monitoring and controlling the performance of the piezoelectric element. The system 700 includes a rechargeable battery 702 that is configured to store electrical energy that can be used to drive the piezoelectric element 714. A piezoelectric driver 708 is electrically connected to the battery 702 and to the piezoelectric element 714. The piezoelectric driver 708 includes electronic circuitry (e.g., analog and/or digital electronic circuitry) that is configured for receiving electrical energy from the battery 702 and for generating a waveform of electrical energy that is provided to the piezoelectric element to drive the piezoelectric element.

In some implementations, the battery 702 can provide electrical energy at a maximum voltage of 5 V or less, for example, at a maximum of 4.4 V or less to the piezoelectric driver 708. The driver 708 can step up the voltage and can output a waveform having a peak-to-peak voltage of greater than 50 V, for example, 100 V, to the piezoelectric element 714. In some implementations, the driver 708 can include step up transformer circuitry configured for receiving a first voltage signal from the battery 702 and for outputting a second voltage signal to the piezoelectric element, where the second voltage is greater than the first voltage.

When the piezoelectric element 714 is associated with a pump of the implantable inflatable device 100, 200, the driver 708 can output a periodic waveform that is used to repeatedly change a volume of a fluid chamber to cause fluid to be pumped through the fluid chamber from one location to another, for example, from a reservoir to an inflatable member or from the inflatable member to the reservoir. In some implementations, the frequency of the periodic waveform can be between 30 Hz and 60 Hz, for example, 40-50 Hz. In some implementations, the periodic waveform can be a sine wave. In some implementations, the periodic waveform can include a series of square pulses. In some implementations, the periodic waveform can include a repeated series of waves provided to the piezoelectric element 714, where the waves have a voltage that varies over time according to a function V=V(t) and where, unlike a sine wave, the second derivative of V divided by V (i.e., V″(t)/V(t)) is not equal to one but where, unlike a square wave, V(t) does not include discontinuities, at which the first derivative of V(t) approaches infinity. A controller or processor 716 can control operations of the piezo driver 708 to control operations of the device.

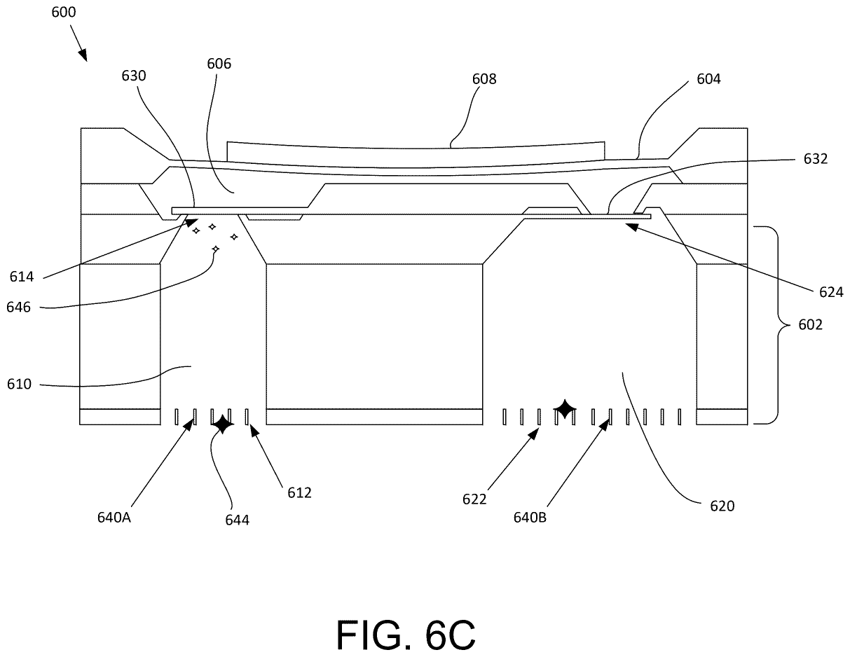

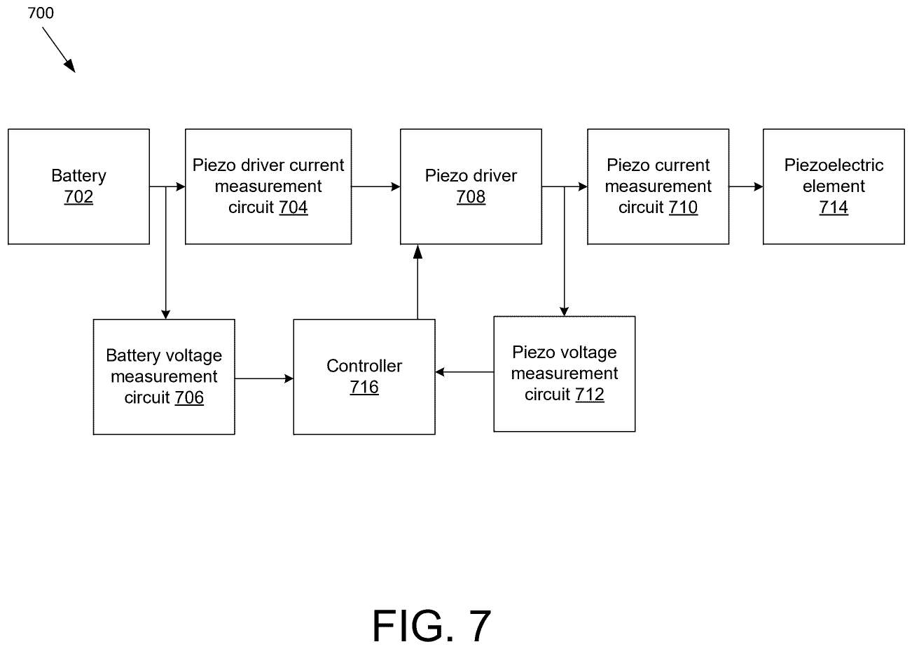

FIG. 8A is a graph of the voltage amplitude of an example waveform that can be a provided by the driver 708 to the piezoelectric element 714 to drive the piezoelectric element to cause a pump associated with the piezoelectric element to pump fluid. The waveform has an amplitude that varies over time according to a function V(t) that is approximated by a sine wave. In the example waveform of FIG. 8A, the voltage varies from −50 V to +50 V and has a frequency of 50 Hz.

FIG. 8B is a graph of the voltage amplitude of another example waveform that can be a provided by the driver 708 to the piezoelectric element 714 to drive the piezoelectric element to cause a pump associated with the piezoelectric element to pump fluid. The waveform has an amplitude that varies over time according to a function V(t) that is approximated by a sine wave having a frequency of 50 Hz. In contrast to the example waveform of FIG. 8A, in the example waveform of FIG. 8B, the average voltage over time is offset from zero, and the voltage varies from −12 V to +88 V. By offsetting the average voltage from zero, a polarization can be induced in the piezoelectric material, which can enhance the mechanical response of the piezoelectric material to the varying voltage of the waveform.

Referring again to FIG. 7, the system 700 can include one or more monitor circuits configured for determining electrical parameters of the waveform that is provided by the driver 708 to the piezoelectric element. For example, a current measurement circuit 710 can measure an electric current drawn by the piezoelectric element 714, and a voltage measurement circuit 712 can measure a voltage of the waveform provided to the piezoelectric element 714, while the piezoelectric element operates to pump fluid in the implantable device. In addition, the system 700 can include one or more monitor circuits configured for determining electrical parameters of electrical energy provided from the battery 702 to the driver 708. For example, a battery voltage measurement circuit 706 can output a measured voltage of the battery 702, and a battery current measurement circuit 704 can measure a current drawn from the battery 702 by the driver 708 while the driver drives the piezoelectric element 714 and powers other components of the system (e.g., a processor, a communication module, etc.).

In some implementations, the battery voltage measurement circuit 706 and the battery current measurement circuit 704 can be used, respectively, to measure the voltage provided by the battery 702 and the current provided by the battery while the piezoelectric-operated pump is used to pump fluid into an inflatable member of the implantable device. After the battery 702 has been fully charged, the current and voltage measurements can be obtained and stored each time the inflatable member is inflated to its designed pressure to determine a state of charge of the battery and to determine a charge capacity of the battery.

Referring again to FIG. 3, the pumps P1, P2 and the valves V1, V2 can be operated in concert with each other to transfer fluid from the reservoir 202 to the inflatable member 204 to inflate the inflatable member and to transfer fluid from the inflatable member 204 to the reservoir 202 to deflate the inflatable member. For example, to inflate the inflatable member 204, a periodic waveform can be applied to P1, V1 can be placed in its open configuration, P2 can be idle, and V2 can be in its closed configuration. To deflate the inflatable member 204, a periodic waveform can be applied to P2, V2 can be placed in its open configuration, P1 can be idle, and V1 can be in its closed configuration. To maintain a pressure in the inflatable member 204, P1 and P2 can be idle, and V1 and V2 can be in their closed configurations.

Referring to FIG. 1, the driver circuitry 108E can include drivers that provide the necessary electrical signals to the piezoelectric elements of pumps P1, P2 and valves V1, V2, so that the valves can open and close and so that the pumps can pump fluid between the reservoir 202 and the inflatable member. The drivers can include a boost circuit that steps up a voltage received from the battery 108D and outputs a higher voltage signal to a pump P1, P2 or valve V1, V2. In some implementations, the drivers can output a voltage of about 88 V to place a valve in its closed configuration and can output a voltage of about-12 V to place the valve in its open configuration. In some implementations, the drivers can output a periodic waveform having a peak-to-peak amplitude of about 100 V to a pump to cause the pump to repeatedly change a volume of a fluid chamber to pump fluid between the reservoir 202 and the inflatable member 204.

Application of a voltage to a piezoelectric element of a pump or valve can deform the piezoelectric from its neutral, or unbiased, shape. Likewise, when a piezoelectric element returns to its neutral, or unbiased, shape from a deformed configuration it can generate a voltage, which can be used to recover energy from the piezoelectric element, for example, in the form of charge that is stored or in the form of a current that drives a load. Techniques are described herein for capturing and using such recovered energy, for example, in manners that do not degrade the lifetime of the rechargeable battery 108D.

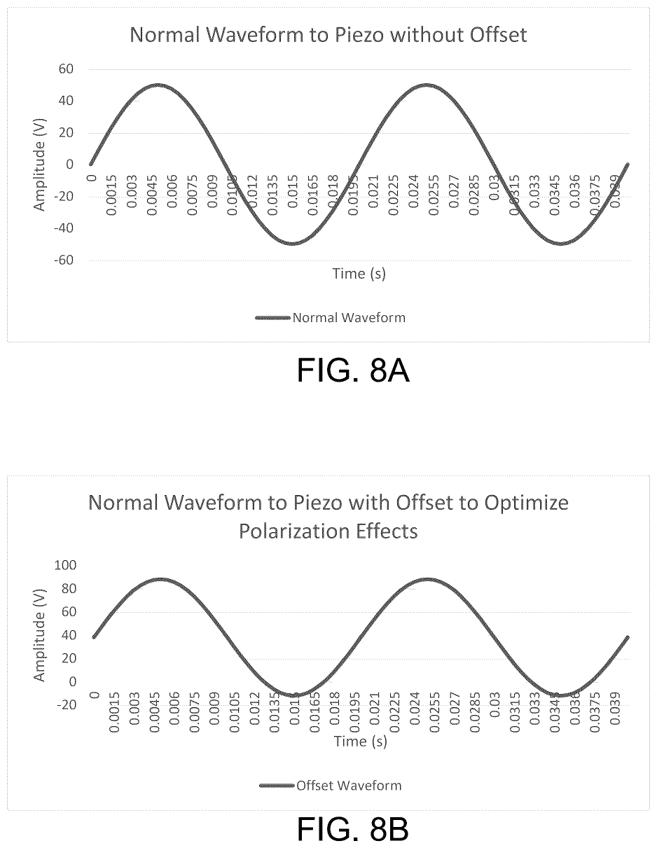

FIG. 9 is an example schematic diagram of a system 900 for driving electrically-operated pumps and valves of an implantable device including an inflatable member and for recovering energy from the pumps and valves to use for driving one or more of the pumps and valves. The system includes a plurality of drivers 910A, 910B, 910C, 910D that are configured for driving piezoelectric elements of the pumps and valves. For example, driver 910A is electrically coupled to a piezoelectric element of a first pump (P1) and includes driver circuitry 912 configured to drive the piezoelectric element to cause the pump to transfer fluid from a reservoir to an inflatable member. Driver 910B is electrically coupled to a piezoelectric element of a first valve (V1) and includes driver circuitry 912 configured to drive the piezoelectric element to open and close the valve, so that the valve can be used to allow or prevent the transfer of fluid from the reservoir to the inflatable member. Driver 910C is electrically coupled to a piezoelectric element of a second pump (P2) and includes driver circuitry 912 configured to drive the piezoelectric element to cause the pump to transfer fluid from the inflatable member to the reservoir. Driver 910D is electrically coupled to a piezoelectric element of a second valve (V2) and includes driver circuitry 912 configured to drive the piezoelectric element to open and close the valve, so that the valve can be used to allow or prevent the transfer of fluid from the inflatable member to the reservoir.

The system 900 includes a rechargeable battery 902 that powers the drivers 910A-D that drive the piezoelectric elements of the pumps P1, P2 and valves V1, V2. The rechargeable battery 902 can be charged by an external charger, as described above, and can supply current to the drivers 910A-D at a voltage of about 3.6 volts (e.g., a voltage greater than 3.0 volts and less than 5.5 volts).

The drivers 910A-D also can include internal circuitry that is configured to harvest electrical energy from the piezoelectric elements associated with the pumps P1, P2 and valves V1, V2, for example, by implementing bidirectional power transfer that can transfer energy from the input of the driver to the output of the driver or from the output to the input. This enables the recovery of the energy from the piezoelectric elements, so that the recovered energy can be transferred back to the input of the driver. An internal controller of the driver 910A-D automatically determines the direction of the power flow while a waveform is output from the driver to a load (e.g., P1, P2, V1, V2) (e.g., on the OUT+ and OUT− pins of the driver). The internal controller includes a Unidirectional Power Input (UPI) switch that toggles whether recovered charge is output to a first input/output pin (VBUS) or to a second input/output pin (VDDP). In the circuit shown in FIG. 9, whether recovered charge is output to a first input/output pin (VBUS) or to a second input/output pin (VDDP) is immaterial, because both pins are connected to one or more capacitors 908, which is/are connected the input to the driver on pin VBUS. Consequently, charge that is output on either the first pin or the second pin is stored on the one or more capacitors and then used to power one or more of the drivers 910A-D.

Charge that is output from a driver 910A-D and stored on the one or more capacitors 908 is prevented from charging the battery 902. For example, protection circuitry 904, which can include a diode or a load switch, is connected in series between the battery 902 and the one or more capacitors to prevent the second circuitry from charging the rechargeable battery 902. This prevents the battery from being charged with energy recovered by the drivers 910A-D from the piezoelectric elements of the pumps and valves and thereby mitigates the deleterious effect on the rechargeable battery of enduring many micro charging events. A clamping diode 906 is connected in parallel with the one or more capacitors 908 to ensure that a voltage level on the capacitors 908 and on the input to the drivers 910A-D does not exceed a threshold voltage. The clamping voltage of the clamping diode 906 can be selected, such that a voltage on capacitor 908 and on first input/output pin (VBUS) does not exceed a threshold value. In some implementations, the threshold value can be determined by the requirements of the driver circuitry 912 of the drivers 910A-D. For example, the threshold value may be less than or equal to 5.5 V. In some implementations, the threshold value can be greater than or equal to 3.0 V, so that the capacitor 908 can be charged to voltage that can be used to power the driver circuitry 912 of the drivers 910A-D.

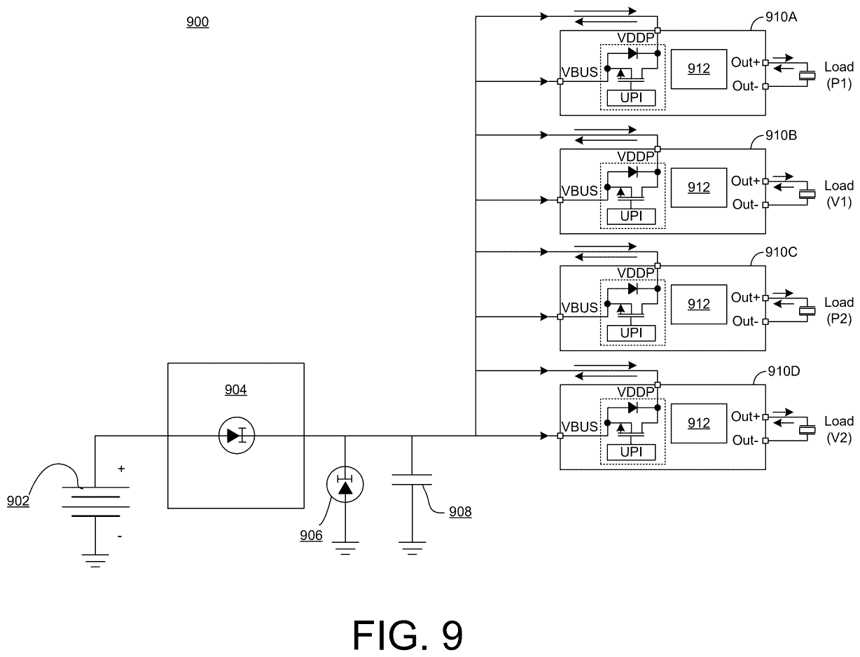

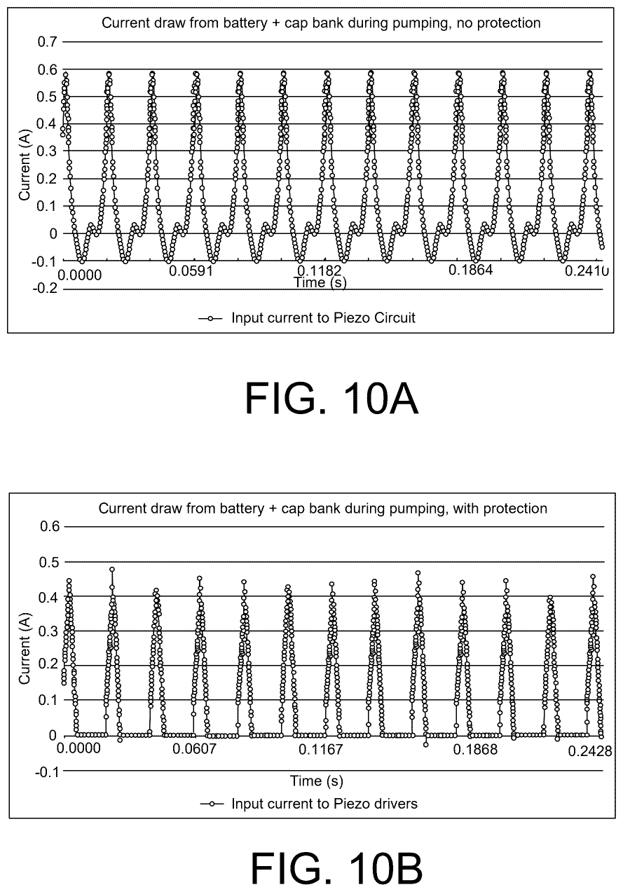

FIG. 10A is an example graph of a current as a function of time, which is drawn from the rechargeable battery 902 by a driver 910A-D that drives a piezoelectric pump when there is no protection circuit 904 between the battery and the driver but when the driver 910A-D operates to recapture energy from the piezoelectric pump and deliver the recaptured energy to the input of the driver. As seen from the graph in FIG. 10A, the periodic waveform that is applied to the piezoelectric element of the pump causes a periodic current draw at about 50 Hz from the battery. The maximum amplitude of the current is about 0.6 Amps, and the current also goes below 0 volts in each cycle, indicating that the battery is being recharged during each 50 Hz cycle of the piezoelectric pump.

FIG. 10B is an example graph of a current as a function of time, which is drawn from the rechargeable battery 902 by a driver 910A-D that drives a piezoelectric pump when the protection circuit 904 is connected between the battery and the driver and when the driver 910A-D operates to recapture energy from the piezoelectric pump and deliver the recaptured energy to the capacitor that is electrically connected to the input of the driver. As seen from the graph in FIG. 10B, the periodic waveform that is applied to the piezoelectric element of the pump causes a periodic current draw at about 50 Hz from the battery. The maximum amplitude of the current is about 0.5 Amps, which is lower than the 0.6 Amp amplitude in FIG. 10A, because, in addition to charge from the battery 902, charge stored on the capacitor 908 is available to supply the driver, thus lowering the current drawn from the battery. In addition, because of the protection circuit 904 that blocks charge from going back into the battery, the current does not go below 0 volts in each cycle, indicating that the battery is not being recharged with energy recovered from the piezoelectric pump.

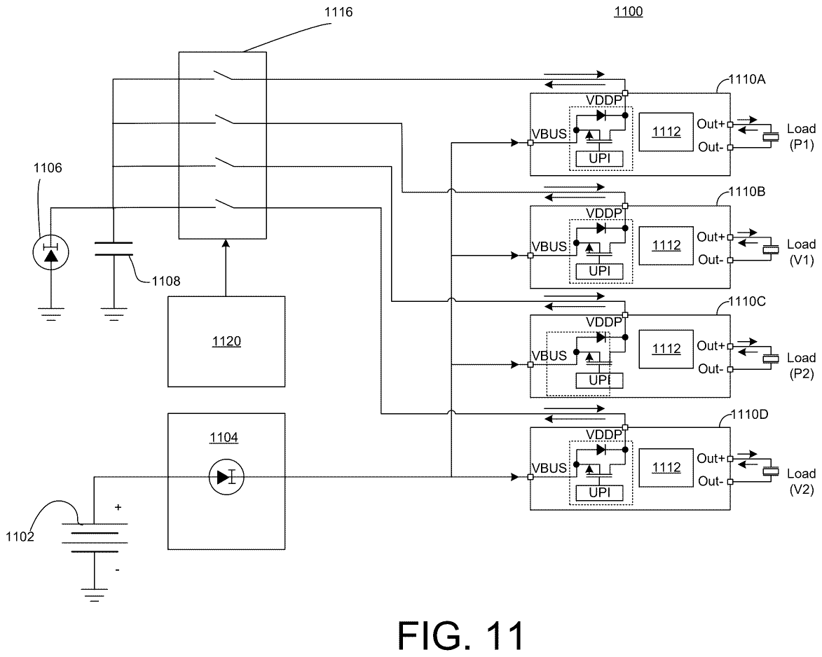

FIG. 11 is an example schematic diagram of a system 1100 for driving electrically-operated pumps and valves of an implantable device including an inflatable member and for recovering energy from the pumps and valves to use for driving one or more of the pumps and valves. The system includes a plurality of drivers 1110A, 1110B, 1110C, 1110D that are configured for driving piezoelectric elements of the pumps and valves. For example, driver 1110A is electrically coupled to a piezoelectric element of a first pump (P1) and includes driver circuitry 1112 configured to drive the piezoelectric element to cause the pump to transfer fluid from a reservoir to an inflatable member. Driver 1110B is electrically coupled to a piezoelectric element of a first valve (V1) and includes driver circuitry 1112 configured to drive the piezoelectric element to open and close the valve, so that the valve can be used to allow or prevent the transfer of fluid from the reservoir to the inflatable member. Driver 1110C is electrically coupled to a piezoelectric element of a second pump (P2) and includes driver circuitry 1112 configured to drive the piezoelectric element to cause the pump to transfer fluid from the inflatable member to the reservoir. Driver 1110D is electrically coupled to a piezoelectric element of a second valve (V2) and includes driver circuitry 1112 configured to drive the piezoelectric element to open and close the valve, so that the valve can be used to allow or prevent the transfer of fluid from the inflatable member to the reservoir.

The system 1100 includes a rechargeable battery 1102 that powers the drivers 1110A-D that drive the piezoelectric elements of the pumps P1, P2 and valves V1, V2. The rechargeable battery 1102 can be charged by an external charger, as described above, and can supply current to the drivers 1110A-D at a voltage of about 3.6 volts (e.g., a voltage greater than 3.0 volts and less than 5.5 volts).

The drivers 1110A-D also can include internal circuitry that is configured to harvest electrical energy from the piezoelectric elements associated with the pumps P1, P2 and valves V1, V2, for example, by implementing bidirectional power transfer that can transfer energy from the input of the driver to the output of the driver or from the output to the input. This enables the recovery of the energy from the piezoelectric elements, so that the recovered energy can be transferred back to the input of the driver. An internal controller of the driver 1110A-D automatically determines the direction of the power flow while a waveform is output from the driver to a load (e.g., P1, P2, V1, V2) (e.g., on the OUT+ and OUT-pins of the driver). The internal controller includes a Unidirectional Power Input (UPI) switch that toggles whether recovered charge is output to a first input/output pin (VBUS) or to a second input/output pin (VDDP). In the circuit shown in FIG. 11, the UPI switches are set to output recovered charge to the second input/output pins (VDDP), which are connected, by way of switches 1116, to one or more capacitors 1108. In some implementations, the one or more capacitors 1108 includes a bank of at least 10 capacitors, with the total capacitance of the bank being greater than or equal to a millifarad.

A processor 1120 can control the operation of the switches 1116 that connect the drivers 1110A-D to the one or more capacitors 1108, so that energy recovered from a piezoelectric element of a pump or valve by a driver 1110A-D can be stored on the one or more capacitors 1108 and can be provided to one or more of the drivers at desired times during the operation of the system 1100. For example, when a driver 1110A or 1110C operates to drive the piezoelectric element of a pump P1 or P2, the switch associated with the driver can be closed during portions of the pumping cycles in which energy is recovered from the piezoelectric element, and opened during portions of the pumping cycles in which energy is provided to the driver from the battery 1102. In this manner, energy is recovered from the piezoelectric element of the pump P1 or P2 and stored on the capacitor(s) 1108, but the recovered energy is not used to drive the piezoelectric element of the pump P1 or P2 in this example implementation.

Instead, the recovered energy stored on the capacitor(s) 1108 can be used for opening and/or closing of the valves V1, V2. When drivers 1110B, 1110D operate to drive the valves V1, V2, the switches 1116 associated with the drivers can be closed, so that energy can be provided by the one or more capacitors 1108 can be used by the drivers to drive the piezoelectric elements of the valves. The energy provided by the one or more capacitors 1108 may be sufficient to drive the piezoelectric elements of the valves or can supplement the energy provided by the battery 1102 to drive the valves.

Charge that is output from a driver 1110A-D and stored on the one or more capacitors 1108 is prevented from charging the battery 1102 because the output from pin VDDP is not connected to the battery 1102. Additionally, optional protection circuitry 1104, which can include a diode or a load switch, can be connected in series between the battery 1102 and the one or more capacitors to prevent the second circuitry from charging the rechargeable battery 1102. A clamping diode 1106 is connected in parallel with the one or more capacitors 1108 to ensure that a voltage level on the capacitors 1108 and on the input to the drivers 1110A-D does not exceed a threshold voltage.

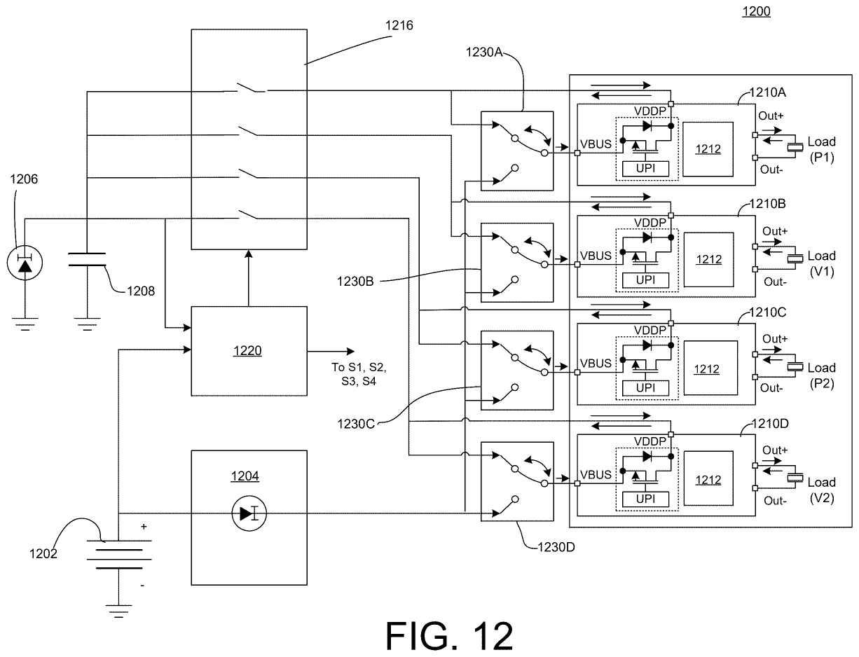

In some implementations, whether energy provided to the drivers comes from the capacitors or from the battery can be controlled explicitly by a processor. For example, FIG. 12 is an example schematic diagram of a system 1200 that includes processor-controlled switches 1230A-D that couple the VBUS pins of drivers 1210A-D to the battery 1202 or to the one or more capacitors 1208. The system 1200 includes a plurality of drivers 1210A, 1210B, 1210C, 1210D that are configured for driving piezoelectric elements of the pumps and valves. For example, driver 1210A is electrically coupled to a piezoelectric element of a first pump (P1) and includes driver circuitry 1212 configured to drive the piezoelectric element to cause the pump to transfer fluid from a reservoir to an inflatable member. Driver 1210B is electrically coupled to a piezoelectric element of a first valve (V1) and includes driver circuitry 1212 configured to drive the piezoelectric element to open and close the valve, so that the valve can be used to allow or prevent the transfer of fluid from the reservoir to the inflatable member. Driver 1210C is electrically coupled to a piezoelectric element of a second pump (P2) and includes driver circuitry 1212 configured to drive the piezoelectric element to cause the pump to transfer fluid from the inflatable member to the reservoir. Driver 1210D is electrically coupled to a piezoelectric element of a second valve (V2) and includes driver circuitry 1212 configured to drive the piezoelectric element to open and close the valve, so that the valve can be used to allow or prevent the transfer of fluid from the inflatable member to the reservoir.

The system 1200 includes a rechargeable battery 1202 that powers the drivers 1210A-D that drive the piezoelectric elements of the pumps P1, P2 and valves V1, V2. The rechargeable battery 1202 can be charged by an external charger, as described above, and can supply current to the drivers 1210A-D at a voltage of about 3.6 volts (e.g., a voltage greater than 3.0 volts and less than 5.5 volts).

The drivers 1210A-D also can include internal circuitry that is configured to harvest electrical energy from the piezoelectric elements associated with the pumps P1, P2 and valves V1, V2, for example, by implementing bidirectional power transfer that can transfer energy from the input of the driver to the output of the driver or from the output to the input. This enables the recovery of the energy from the piezoelectric elements, so that the recovered energy can be transferred back to the input of the driver. An internal controller of the driver 1210A-D automatically determines the direction of the power flow while a waveform is output from the driver to a load (e.g., P1, P2, V1, V2) (e.g., on the OUT+ and OUT− pins of the driver). The internal controller includes a Unidirectional Power Input (UPI) switch that toggles whether recovered charge is output to a first input/output pin (VBUS) or to a second input/output pin (VDDP). In the circuit shown in FIG. 12, the UPI switches are set to output recovered charge to the second input/output pins (VDDP), which are connected, by way of switches 1216, to one or more capacitors 1208. In some implementations, the one or more capacitors 1208 includes a bank of at least 10 capacitors, with the total capacitance of the bank being greater than or equal to a millifarad.

A processor 1220 can control the operation of the switches 1216 that connect the drivers 1210A-D to the one or more capacitors 1208, so that energy recovered from a piezoelectric element of a pump or valve by a driver 1210A-D can be stored on the one or more capacitors 1208 and can be provided to one or more of the drivers at desired times during the operation of the system 1200. For example, when a driver 1210A or 1210C operates to drive the piezoelectric element of a pump P1 or P2, the switch associated with the driver can be closed during portions of the pumping cycles in which energy is recovered from the piezoelectric element, and opened during portions of the pumping cycles in which energy is provided to the driver from the battery 1202. In this manner, energy is recovered from the piezoelectric element of the pump P1 or P2 and stored on the capacitor(s) 1208, but the recovered energy is not used to drive the piezoelectric element of the pump P1 or P2.

The processor 1220 also can control how the recovered energy stored on the capacitor(s) 1208 is allocated to the drivers 1210A-D to power the piezoelectric elements connected to the drivers. For example, the processor 1220 can control switches 1230A, 1230B, 1230C, 1230D to determine whether pin VBUS of a driver is connected to the capacitor(s) 1208, in which case the driver is powered only by the energy stored on the one or more capacitors or whether the VBUS pin is connected to the battery 1202, in which case the driver can be powered by energy stored on the one or more capacitors 1208 and in the battery 1202. Voltage measurements of the capacitor and the battery can be provided to the processor 1220, and the processor can control the switches 1230A-D based on the voltage measurements. For example, when a voltage measurement from the battery 1202 indicates that the battery is nearing the end of its charge, or when a voltage measurement of a capacitor indicates the capacitor has a very high state of charge, the processor 1220 may control the switches 1230A-D to prioritize the provision of energy from the capacitor(s) 1208 to the drivers. In another example, when a voltage measurement from the battery 1202 indicates that the battery is close to fully charged, or when a voltage measurement of a capacitor indicates the capacitor has a very low state of charge, the processor 1220 may control the switches 1230A-D to prioritize the provision of energy from the battery 1202 to the drivers.

Charge that is output from a driver 1210A-D and stored on the one or more capacitors 1208 is prevented from charging the battery 1202, because pins VDDP are not connected to the battery 1202. In addition, optional protection circuitry 1204, which can include a diode or a load switch, can be connected in series between the battery 1202 and the one or more capacitors to prevent the second circuitry from charging the rechargeable battery 1202. A clamping diode 1206 is connected in parallel with the one or more capacitors 1208 to ensure that a voltage level on the capacitors 1208 and on the input to the drivers 1210A-D does not exceed a threshold voltage.

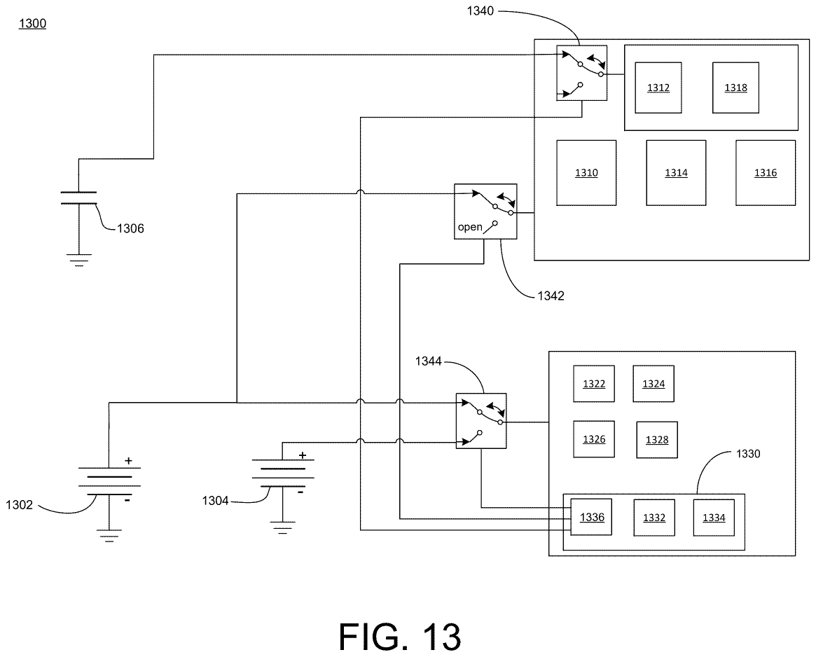

FIG. 13 is an example schematic diagram of an implantable fluid-operated medical device system 1300 that includes a plurality of different energy storage devices that are configured for storing energy and providing electrical energy to one or more electrical circuits of the system. For example, the system 1300 can include a rechargeable battery 1302, a non-rechargeable battery 1304, and one or more capacitors 1306, each of which is configured for storing energy that can be provided to the circuits of the system 1300.

In some implementations, the rechargeable battery 1302 may be susceptible to damage if, during operation of the system 1300, the state of charge on the rechargeable battery falls below a threshold value or if the state of charge falls below the threshold value more than a predetermined number of times. For example, rechargeable lithium-ion batteries typically operate within a predetermined voltage range (e.g., 3.0V to 4.2V), and discharging a rechargeable lithium-ion below the minimum threshold voltage value (e.g., around 2.5V to 3.0V) can lead to irreversible chemical reactions that damage the battery. For example, if the state of charge of a rechargeable lithium-ion battery falls below a minimum threshold voltage value, a significant reduction of the battery's energy storage capacity result from a dissolution of the substrate and film of a solid electrolyte interface of the battery. If the rechargeable battery 1302 falls below the minimum threshold voltage value by too much or more than a particular number of times, the rechargeable battery may not recover and may only be able to provide an output voltage of 0 V or close to 0 V.

Therefore, to protect the functionality of the rechargeable battery 1302 and of the system 1300, the system can include the non-rechargeable battery 1304, so that, when a state of charge of the rechargeable battery 1302 falls below a threshold value, the rechargeable battery 1302 can the decoupled from providing electrical energy to certain electrical circuits of the system, but critical circuits of the system still can be operated in a low-power mode with the provision of the electrical energy from the non-rechargeable battery 1304.

The system 1300 includes a plurality of electrical circuits, which can have different requirements of electrical energy to operate. For example, an electrical circuit 1310 that drives the operation of a piezoelectric pump may require more energy to inflate or deflate an inflatable member than an electrical circuit 1312 that drives the operation of a piezoelectric valve to open or close the valve. Similarly, an electrical circuit 1314 that controls the operation of communicating with an external device outside a patient's body (e.g., a Bluetooth® communication circuit) and an electrical circuit 1316 that controls the operation of, and the reception of data from, an accelerometer of the system 1300 may require more energy to perform their operations than an electrical circuit 1318 that controls the operation of, and the reception of data from, a pressure sensor of the system. A first group of electrical circuits (e.g., electrical circuits 1312, 1318), which may have relatively low energy requirements, can be classified as electrical circuits that are powered primarily with energy stored on the one or more capacitors 1306, but which can be powered with energy stored in the rechargeable battery 1302 when a state of charge of the one or more capacitors 1306 is below a threshold value.

In addition, the system 1300 includes a plurality of electrical circuits that can be categorized as tolerant or non-tolerant to the lack of provision of electrical energy from the energy storage devices 1302, 1304, 1306. For example, a second group of electrical circuits 1310, 1312, 1314, 1316, 1318 that drive the operation of piezoelectric elements of one or more piezoelectric pumps and piezoelectric valves, that drive and control the operation of one or more accelerometers and pressure sensors, and that drive the operation of a primary communication circuit for communicating with the external device can be categorized as tolerant to the lack of provision of electrical energy from the energy storage devices 1302, 1304, 1306. The second group of electrical circuits can include one or more electrical circuits of the first group of electrical circuits. In some implementations, the second group of electrical circuits can be powered by energy stored on at least one of the rechargeable battery 1302 and the at least one capacitor 1306.

A third group of the electrical circuits, for example, electrical circuits 1322, 1324, 1326, 1328 can be categorized as non-tolerant to the lack of provision of electrical energy from the energy storage devices, such that they require an uninterrupted supply of electrical energy from at least one of the energy storage devices 1302, 1304, 1306. In some implementations, the third group of electrical circuits can include, for example, an integrated circuit 1330, for example, an application specific integrated circuit that includes a processor 1332 and a memory device 1334, and which controls and coordinates the operations of the system 1300. In some implementations, the third group of electrical circuits can include, for example, one or more voltage monitoring circuits 1322 configured for monitoring a voltage on, or a current delivered from, one or more of individual energy storage devices 1302, 1304, 1306. In some implementations, the third group of electrical circuits can include, for example, a wireless power transmission control circuit 1324 that receives energy from an external power transmission device and provides the energy to charge the rechargeable battery 1302. In some implementations, the third group of electrical circuits can include, for example, a low-power mode communication circuit 1326, which is configured for sending and/or receiving communication signals to/from an external controller, where the communication signals, may, for example, alert the external controller about a low-power state of the rechargeable battery 1302, wake up the wireless power transmission control circuit 1324 to receive electrical energy to recharge the rechargeable battery 1302, etc. In some implementations, the third group of electrical circuits can include, for example, a low-power mode valve control circuit 1328, which can drive the operation of one or more piezoelectric valves of the system 1300. The low-power mode valve control circuit 1328 can be used, for example, to open or close a piezoelectric valve so that fluidic pressure in an inflatable member of the system 1300 can be maintained or relieved, even when the rechargeable battery 1302 has a state of charge that is below a threshold value.

The integrated circuit 1330 can include a switch controller 1336 that is configured to control the operation of one or more switches 1340, 1342, 1344 that are operable to switch the provision of the electrical energy to a group of electrical circuits between one or more of the energy storage devices 1302, 1304, 1306. For example, the switch controller 1336 can control switch 1342 to switch the provision of electrical energy to the first group of electrical circuits between the at least one capacitor 1306 and the rechargeable battery 1302. The switch 1340 can be controlled based on a state of charge of the at least one capacitor. For example, switch 1340 can be controlled to provide electrical energy to the first group of electrical circuits from the at least one capacitor when the state of charge on the at least one capacitor exceeds a first threshold value and to provide electrical energy to the first group of electrical circuits from a rechargeable battery 1302 when the state of charge on the at least one capacitor is less than the first threshold value.

In another example, switch 1342 can be controlled to provide electrical energy to the second group of electrical circuits from the rechargeable battery 1302 only when a state of charge of the rechargeable battery exceeds a threshold value. For example, when the voltage that can be provided by the rechargeable battery 1302 is lower than a threshold voltage (e.g., lower than 3.0 V, lower than 2.5 V, or lower than 2.0 V) the rechargeable battery 1302 can be placed into a hibernation mode by using the switch 1342 to disconnect the rechargeable battery 1302 from the second group of electrical circuits. In the low-power hibernation mode, high-power draw electrical circuits, for example, the electrical circuit 1310 that drives a piezoelectric pump that inflates or deflates an inflatable member can be turned off to preserve charge on the rechargeable battery 1302. Thus, in the low-power hibernation mode, the rechargeable battery 1302 maintains its limited amount of charge above a threshold value below which the rechargeable battery could be damaged.

In another example, switch 1344 can be controlled to provide electrical energy to the third group of electrical circuits from the rechargeable battery 1302 only when a state of charge of the rechargeable battery exceeds a threshold value and to provide electrical energy to the third group of electrical circuits from the non-rechargeable battery 1304 when the state of charge of the rechargeable battery is less than the threshold value. In this manner, critical electrical circuits of the third group can be continuously supplied with electrical energy, even when a state of charge of the rechargeable battery 1302 falls below the threshold value and the system 1300 enters a hibernation mode in which the rechargeable battery is disconnected from the second group of electrical circuits. As described above, the electrical circuits of the third group can include an integrated circuit 1330 that includes a processor 1332 that is essential for controlling the operations of the system 1300, even when the system has entered the hibernation mode and which includes one or more memory devices 1334, for example, non-volatile memory that stores executable instructions and that requires electrical energy to maintain the executable instructions. The third group of electrical circuits also can include a low-power mode communication circuit 1326 that can be used to communicate simple instructions between the system 1300 and an external device in a manner that uses less power than the communication circuit 1314. In an example implementation, the low-power mode communication circuit 1326 can include a Hall effect sensor that can receive signals from an external controller. The external controller can provide the signals by placing a magnet in proximity to the system 1300 to generate an inductive current in the low-power mode communication circuit 1314. Signals received by the low-power mode communication circuit 1326 can cause the integrated circuit 1330 to, for example, wake up a wireless power transmission control circuit 1324 to begin charging the rechargeable battery 1302 or cause the low-power mode valve control circuit 1328 to open or close a piezoelectric valve so that fluidic pressure in an inflatable member of the system 1300 the maintained or relieved, even when the rechargeable battery 1302 has a state of charge that is below a threshold value.

While certain features of the described implementations have been illustrated as described herein, many modifications, substitutions, changes and equivalents will now occur to those skilled in the art. It is, therefore, to be understood that the appended claims cover all such modifications and changes as fall within the scope of the embodiments.

What is claimed is:

1. An implantable fluid-operated device configured to control fluid flow between a fluid reservoir and an inflatable member, the device comprising:
a rechargeable battery configured for storing energy;
energy transmission circuitry configured for receiving energy from an external power transmission device and for providing energy to charge the rechargeable battery;
a non-rechargeable battery configured for storing energy;
at least one capacitor configured for storing electrical charge;
a first piezoelectric pump configured to transfer fluid from the fluid reservoir to the inflatable member;
a first driver including first circuitry configured for providing a waveform of electrical energy from the rechargeable battery to a piezoelectric element of the first piezoelectric pump to drive the first piezoelectric to pump fluid from the fluid reservoir to the inflatable member, and second circuitry configured for charging the at least one capacitor in response to a voltage generated by the piezoelectric element when the piezoelectric element returns to its neutral shape from a deformed shape;
a first group of at least one first electrical circuits;
a second group of at least one second electrical circuits;
a third group of at least one third electrical circuits; and
a processor that is configured to execute instructions that cause the implantable fluid-operated device to:
switch a provision of electrical energy to the first group between the at least one capacitor and the rechargeable battery based on a state of charge of the at least one capacitor;
control whether electrical energy is provided to the second group from the rechargeable battery based on a state of charge of the rechargeable battery; and
switch the provision of electrical energy to the third group between the rechargeable battery and the non-rechargeable battery based on a state of charge of the rechargeable battery.

2. The implantable fluid-operated device of claim 1, wherein the processor is configured to execute instructions that cause the implantable fluid-operated device to provide electrical energy to the first group from the at least one capacitor when a state of charge on the at least one capacitor exceeds a first threshold and to provide electrical energy to the first group from the rechargeable battery when the state of charge is less than the first threshold.

3. The implantable fluid-operated device of claim 1, wherein the processor is configured to execute instructions that cause the implantable fluid-operated device to provide electrical energy to the second group from the rechargeable battery when a state of charge of the rechargeable battery exceeds a second threshold and to not provide electrical energy to the second group from the rechargeable battery when the state of charge of the rechargeable battery is less than the second threshold.

4. The implantable fluid-operated device of claim 3, wherein the processor is configured to execute instructions that cause the implantable fluid-operated device to provide electrical energy to the second group and to the third group from the rechargeable battery when a state of charge of the rechargeable battery exceeds the second threshold and to not provide electrical energy to the second group from the rechargeable battery when the state of charge of the rechargeable battery is less than the second threshold and to provide electrical energy to the third group from the non-rechargeable battery when the state of charge of the rechargeable battery is less than a third threshold.

5. The implantable fluid-operated device of claim 4, wherein the third threshold is equal to the second threshold.

6. The implantable fluid-operated device of claim 1, wherein the first driver and the second circuitry belong to the second group.

7. The implantable fluid-operated device of claim 1, wherein the second group includes the first group.

8. The implantable fluid-operated device of claim 1, wherein implantable fluid-operated device includes a penile implant, and the implantable fluid-operated device further comprises:
a second piezoelectric pump configured to transfer fluid from the inflatable member to the fluid reservoir;
a second driver including third circuitry configured for providing a waveform of electrical energy from the rechargeable battery to a piezoelectric element of the second piezoelectric pump to drive the second piezoelectric to pump fluid from the inflatable member to the fluid reservoir, and fourth circuitry configured for charging the at least one capacitor in response to a voltage generated by the piezoelectric element when the piezoelectric element returns to its neutral shape from a deformed shape; and
a protection circuit configured to prevent the fourth circuitry from charging the rechargeable battery, wherein the second driver and the third and fourth circuitry belong to the second group.

9. The implantable fluid-operated device of claim 8, further comprising:
one or more valve circuits configured for opening and closing one or more fluidic valves that control the flow of fluid between the fluid reservoir and the inflatable member, wherein the one or more valve circuits belong to the first group.

10. The implantable fluid-operated device of claim 1, wherein the inflatable member includes an inflatable cuff configured for implantation about a urethra of a patient, and the implantable fluid-operated device further comprises:
a second piezoelectric pump configured to transfer fluid from the inflatable member to the fluid reservoir;
a second driver including third circuitry configured for providing a waveform of electrical energy from the rechargeable battery to a piezoelectric element of the second piezoelectric pump to drive the second piezoelectric to pump fluid from the inflatable member to the fluid reservoir, and fourth circuitry configured for charging the at least one capacitor in response to a voltage generated by the piezoelectric element when the piezoelectric element returns to its neutral shape from a deformed shape; and
a protection circuit configured to prevent the fourth circuitry from charging the rechargeable battery, wherein the second driver and the third and fourth circuitry belong to the second group;
one or more valve circuits configured for opening and closing one or more fluidic valves that control the flow of fluid between the fluid reservoir and the inflatable member, wherein the one or more valve circuits belong to the first group and to the third group.

11. The implantable fluid-operated device of claim 10, further comprising:
a Bluetooth® communication circuit that belongs to the second group; and
a low power communication circuit configured for sending information to, and for receiving information from, an external controller, wherein the low power communication circuit belongs to the third group.

12. A method of operating an implantable fluid-operated device that controls fluid flow between a fluid reservoir and an inflatable member, the implantable fluid-operated device including a rechargeable battery, a non-rechargeable battery, at least one capacitor, a first piezoelectric pump configured to transfer fluid from the fluid reservoir to the inflatable member, the method comprising:
providing, with a first driver that includes first circuitry, a waveform of electrical energy from the rechargeable battery to a piezoelectric element of the first piezoelectric pump to drive the first piezoelectric pump to pump fluid from the fluid reservoir to the inflatable member;
charging, with second circuitry, the at least one capacitor in response to a voltage generated by the piezoelectric element when the piezoelectric element returns to its neutral shape from a deformed shape;
switching a provision of electrical energy to a first group of at least one first electrical circuits from the at least one capacitor to the rechargeable battery based on a state of charge of the at least one capacitor;
controlling whether electrical energy is provided to a second group of at least one second electrical circuits from the rechargeable battery based on a state of charge of the rechargeable battery; and
switching the provision of electrical energy to a third group of at least one third electrical circuits from the rechargeable battery to the non-rechargeable battery based on a state of charge of the rechargeable battery.

13. The method of claim 12, further comprising:
providing electrical energy to the first group from the at least one capacitor when a state of charge on the at least one capacitor exceeds a first threshold; and
providing electrical energy to the first group from the rechargeable battery when the state of charge is less than the first threshold.

14. The method of claim 12, further comprising:
providing electrical energy to the second group from the rechargeable battery when a state of charge of the rechargeable battery exceeds a second threshold;
blocking the provision of electrical energy to the second group from the rechargeable battery when the state of charge of the rechargeable battery is less than the second threshold.

15. The method of claim 14, further comprising:
providing electrical energy to the second group and to the third group from the rechargeable battery when a state of charge of the rechargeable battery exceeds the second threshold;
blocking the provision of electrical energy to the second group from the rechargeable battery when the state of charge of the rechargeable battery is less than the second threshold;
providing electrical energy to the third group from the non-rechargeable battery when the state of charge of the rechargeable battery is less than a third threshold.

16. The method of claim 15, wherein the third threshold is equal to the second threshold.

17. The method of claim 12, wherein the first driver and the second circuitry belong to the second group, and wherein the second group includes the first group.

18. The method of claim 12, wherein the implantable fluid-operated device includes a penile implant, and further includes a second piezoelectric pump configured to transfer fluid from the inflatable member to the fluid reservoir, the method further comprising:
providing, with a second driver that includes third circuitry, a waveform of electrical energy from the rechargeable battery to a piezoelectric element of the second piezoelectric pump to drive the second piezoelectric to pump fluid from the inflatable member to the fluid reservoir, wherein the second driver and the third and fourth circuitry belong to the second group;
charging, with fourth circuitry, the at least one capacitor in response to a voltage generated by the piezoelectric element when the piezoelectric element returns to its neutral shape from a deformed shape; and
preventing the fourth circuitry from charging the rechargeable battery.

19. The method of claim 18, wherein the implantable fluid-operated device includes one or more valve circuits configured for opening and closing one or more fluidic valves that control the flow of fluid between the fluid reservoir and the inflatable member, wherein the one or more valve circuits belong to the first group.

20. The method of claim 12, wherein the inflatable member includes an inflatable cuff configured for implantation about a urethra of a patient, wherein the implantable fluid-operated device further includes a second piezoelectric pump configured to transfer fluid from the inflatable member to the fluid reservoir, the method further comprising:
providing, by a second driver including third circuitry, a waveform of electrical energy from the rechargeable battery to a piezoelectric element of the second piezoelectric pump to drive the second piezoelectric to pump fluid from the inflatable member to the fluid reservoir, wherein the second driver belongs to the second group;
charging, with fourth circuitry, the at least one capacitor in response to a voltage generated by the piezoelectric element when the piezoelectric element returns to its neutral shape from a deformed shape, wherein the third and fourth circuitry belong to the second group;
controlling, with one or more valve circuits configured for opening and closing one or more fluidic valves, the flow of fluid between the fluid reservoir and the inflatable member, wherein the one or more valve circuits belong to the first group and to the third group; and
preventing the fourth circuitry from charging the rechargeable battery.
